(19) United States
(12) Patent Application Publication
(10) Pub. No.: US 2026/0182846 A1
(43) Pub. Date: Jul. 2, 2026

(54) PATIENT-MONITORING SYSTEM

(71) Applicants: Baxter International Inc., Deerfield, IL (US); Baxter Healthcare SA, Glattpark (Opfikon) (CH)

(72) Inventors: Matthew Bivans, Deerfield, IL (US); Ahren Ceisel, Deerfield, IL (US); Vivek Walimbe, Deerfield, IL (US); Jonathan Handler, Northbrook, IL (US); Marshal Dhillon, San Diego, CA (US); Mark Dhillon, San Diego, CA (US); Erik Tang, San Diego, CA (US); James McCanna, Pleasanton, CA (US); Lauren N.M. Hayward, San Diego, CA (US); Matthew Banet, San Diego, CA (US)

(21) Appl. No.: 19/545,789

(22) Filed: Feb. 20, 2026

Related U.S. Application Data
(62) Division of application No. 17/354,150, filed on Jun. 22, 2021.
(60) Provisional application No. 63/043,494, filed on Jun. 24, 2020.

(51) Int. Cl. A61B 5/0205; A61B 5/00; A61B 5/021; A61B 5/0215

(52) U.S. Cl. CPC A61B 5/0205; A61B 5/021; A61B 5/02152; A61B 5/4839; A61B 5/6852; A61B 5/721; A61B 5/7257; A61B 5/726; A61B 2562/0219; A61B 2562/0247

(57) ABSTRACT

The invention provides an IV system for monitoring a patient that is positioned on the patient's body. The IV system includes: 1) a catheter that inserts into the patient's venous system; 2) a pressure sensor connected to the catheter that measures physiological signals indicating a pressure in the patient's venous system; 3) a motion sensor that measures motion signals; and 4) a processing system that: i) receives the physiological signals from the pressure sensor; ii) receives the motion signals from the motion sensor; iii) processes the motion signals by comparing them to a pre-determined threshold value to determine when the patient has a relatively low degree of motion; and iv) process the physiological signals to determine a physiological parameter when the processing system determines that the motion signals are below the pre-determined threshold value.

### PRIORITY CLAIM AND CROSS-REFERENCE TO RELATED APPLICATIONS

This application is a divisional of U.S. patent application Ser. No. 17/354,150, entitled PATIENT-MONITORING SYSTEM, filed on Jun. 22, 2021, which claims priority to and the benefit of U.S. Provisional Ser. No. 63/043,494 , entitled PATIENT-MONITORING SYSTEM, filed Jun. 24, 2020, the entire contents of which are hereby incorporated by reference in its entirety and relied upon.

### FIELD OF THE INVENTION

The invention described herein relates to systems for drug and fluid delivery, and to systems for monitoring patients in, e.g., hospitals and medical clinics.

### BACKGROUND

Unless a term is expressly defined herein using the phrase “herein “______””, or a similar sentence, there is no intent to limit the meaning of that term beyond its plain or ordinary meaning. To the extent that any term is referred to in this document in a manner consistent with a single meaning, that is done for sake of clarity only; it is not intended that such claim term be limited to that single meaning. Finally, unless a claim element is defined by reciting the word “means” and a function without the recital of any structure, it is not intended that the scope of any claim element be interpreted based on the application of 35 U.S.C. § 112(f).

Proper care of hospitalized patients typically requires: 1) delivery of medications and fluids using intravenous (herein “IV”) catheters and infusion pumps; and 2) measuring vital signs and hemodynamic parameters with patient monitors. Typically, IV catheters insert in veins in the patient's hands or arms, and patient monitors connect to sensors or electrodes worn on (or inserted in) the patient's body.

Conventional patient monitors typically measure electrocardiogram (herein “ECG”) and impedance pneumography (herein “IP”) waveforms using torso-worn electrodes, from which they calculate heart rate (herein “HR”), heart rate variability (herein “HRV”), and respiration rate (herein “RR”). Most conventional monitors also measure optical signals, called photoplethysmogram (herein “PPG”) waveforms, with sensors that typically clip on the patient's fingers or earlobes. Such sensors can calculate blood oxygen levels (herein “SpO2”) and pulse rate (herein “PR”) from these PPG waveforms. More advanced monitors can also measure blood pressure (herein “BP”), notably systolic (herein “SYS”), diastolic (herein “DIA”), and mean (herein “MAP”) BP, typically using cuff-based techniques called oscillometry, or pressure-sensitive catheters that insert into a patient's arterial system called arterial lines. Digital stethoscopes, which can be either portable and body-worn devices, can measure phonocardiogram (herein “PCG) waveforms that indicate heart sounds and murmurs.

Some patient monitors are entirely body-worn. These typically take the shape of patches that measure ECG, HR, HRV and, in some cases, RR. Such patches can also include accelerometers that measure motion (herein “ACC”) waveforms. Algorithms can determine the patient's posture, degree of motion, falls, and other related parameters from the ACC waveforms. Patients typically wear these types of patches in the hospital or, alternatively, for ambulatory and home use. The patches are typically worn for relatively short periods of time (e.g., from a few days to several weeks). They are typically wireless, and usually include technologies such as Bluetooth® transceivers to transmit information over a short range to a secondary ‘gateway’ device, which typically includes a cellular or Wi-Fi radio to transmit the information to a cloud-based system.

Even more complex patient monitors measure parameters such as stroke volume (herein “SV”), cardiac output (herein “CO”), and cardiac wedge pressure using an invasive sensor called a Swan-Ganz or pulmonary-artery catheter. To make a measurement, these sensors are positioned in the patient's left heart, where they are ‘wedged’ into a small pulmonary blood vessel using a balloon catheter. As an alternative to this highly invasive measurement, patient monitors can use non-invasive techniques such as bio-impedance and bio-reactance to measure similar parameters. These methods deploy body-worn electrodes (typically deployed on the patient's chest, legs, and/or neck) to measure impedance plethysmogram (herein “IPG”) and/or bio-reactance (herein “BR”) waveforms. Analysis of IPG and BR waveforms yields SV, CO, and thoracic impedance, which is a proxy for fluids in the patient's chest (herein “FLUIDS”). Notably, IPG and BR waveforms generally have similar shapes and are sensed using similar measurement techniques, and are thus used interchangeably herein.

Devices that measure sv, co, and fluids can establish a patient's blood volume, fluid responsiveness, and, in some cases, related metrics such as central venous pressure (herein “CVP”). Taken collectively, these parameters can diagnose certain medical conditions and guide resuscitation efforts. But the highly invasive nature of Swan-Ganz and pulmonary-artery catheters can be disadvantageous and comes with a high risk of infection. Additionally, CVP measurements may be slower to change in response to certain acute conditions, such as when the circulatory system attempts to compensate for blood volume disequilibrium (particularly hypovolemia) by protecting blood volume levels in the central circulatory system at the expense of the periphery. For example, constriction in peripheral blood vessels may reduce the effect of fluid loss on the central system, thereby temporarily masking blood loss in conventional CVP measurements. Such masking can lead to delayed recognition and treatment of patient conditions, thereby worsening outcomes.

To address these and other shortcomings, a measurement technique called peripheral intravenous waveform analysis (herein “PIVA”) has been developed, as described in U.S. patent application Ser. No. 14/853,504 (filed Sep. 14, 2015 and published as U.S. Patent Publication No. 2016/0073959) and PCT Application No. PCT/US 16/16420 (filed Feb. 3, 2016, and published as WO 2016/126856), the contents of which are incorporated herein by reference. These documents describe sensors featuring pressure transducers that receive signals from in-dwelling catheters inserted in a patient's venous system, and connect through cables to remote electronics that process signals generated therefrom (herein “PIVA sensor”). PIVA sensors measure time-dependent waveforms indicating peripheral venous pressure (herein “PVP”) using existing IV lines, which typically include IV tubing attached to a saline drip or infusion pump. Measurements made with PIVA sensors typically feature a mathematical transformation of the PVP waveforms into the frequency domain, performed with a remote computer, using a methodology called fast Fourier Transform (herein “FFT”). Analysis of a frequency-domain spectrum generated with an FFT can yield a RR frequency (herein “F0”) and a HR frequency (herein “F1”) indicating, respectively, the patient's HR and RR. A more detailed analysis of F0 and F1, e.g. use of a computer algorithm to determine the amplitude of these peaks or, alternatively, integrate an area underneath the curve centered around the maximum peak amplitude, determines the ‘energy’ of these features. Further processing of these energies yields an indication of a patient's blood volume status. Such measurements have been described, for example, in the following references, the contents of which are herein incorporated by reference: 1) Hocking et al., “Peripheral venous waveform analysis for detecting hemorrhage and iatrogenic volume overload in a porcine model.”, Shock. 2016 October; 46(4): 447-52; 2) Sileshi et al., “Peripheral venous waveform analysis for detecting early hemorrhage: a pilot study.”, Intensive Care Med. 2015 June; 41(6): 1147-8; 3) Miles et al., “Peripheral intravenous volume analysis (PIVA) for quantitating volume overload in patients hospitalized with acute decompensated heart failure-a pilot study.”, J Card Fail. 2018 August; 24(8): 525-532; and 4) Hocking et al., “Peripheral i.v. analysis (PIVA) of venous waveforms for volume assessment in patients undergoing haemodialysis.”, Br J Anaesth. 2017 December 1; 119(6): 1135-1140.

Unfortunately, during typical measurements with PIVA sensors, PVP waveforms induced by HR and RR events (typically 5-20 mmHg) are much weaker than their arterial pressure counterparts (typically 60-150 mmHg). This means magnitudes of corresponding signals in time-dependent PVP waveforms measured by conventional pressure transducers are often very weak (e.g. typically 5-50 V). Additionally, PVP waveforms are typically amplified, conditioned, digitized, and ultimately processed with electronic systems located remotely from the patient. Thus, prior to these steps, analog versions of the waveforms travel through cables that can attenuate them and add noise (due, e.g., to motion). And in some cases, PVP waveforms simply lack signatures corresponding to F0 and F1. Or peaks of one primary frequency are obscured by ‘harmonics’ (i.e. integer multiple of a given frequency) of the other primary frequency. This can make it difficult or impossible for an automated medical device to accurately determine F0 and F1, and the energy associated with these features.

### SUMMARY OF THE INVENTION

In view of the foregoing, it would be beneficial to improve a conventional PIVA sensor so that it overcomes historical problems related to weak, noisy PVP waveforms and inadequate detection of F0 and F1. Such as system could improve how patients are monitored in hospitals and medical clinics. To cure these and other deficiencies, described herein is an augmented, improved PIVA sensor (herein “iPIVA sensor”) featuring: 1) a circuit board located in close proximity to an in-dwelling venous catheter that amplifies, filters, and digitizes PVP waveforms immediately after a pressure sensor detects them (e.g. directly on the patient's body); and 2) a chest-worn physiological sensor (herein “patch sensor”) that makes accurate, independent measurements of vital signs, including HR and RR, which can assist in locating F0 and F1, and then processes these features to determine their corresponding energies. An iPIVA sensor according to the invention can include one or both of these improvements. Additionally, according to the invention, measurements from the iPIVA sensor can be coupled with independent measurements of hemodynamic parameters, e.g. SV, CO, and FLUIDS (which can be made with the patch sensor or a comparable patient monitor) to yield an improved understanding of the patient's fluid status. Ultimately the combination of these technologies—an iPIVA sensor featuring a novel signal-conditioning circuit board combined with a complementary patch sensor that measures both vital signs and hemodynamic parameters—may improve how patients are monitored and resuscitated in hospitals and medical clinics.

The iPIVA sensor described herein is designed to work with a conventional IV system, and connects to the patient with an in-dwelling catheter, both of which are standard equipment. The catheter includes a housing, worn close to or on the patient's body, and typically on their arm or hand, that encloses a signal-conditioning circuit board featuring complex circuitry that amplifies, filters, and digitizes analog PVP waveforms. The circuit board may also include components for processing and storing the digitized signals, measuring motion (e.g. an accelerometer and/or gyroscope), and wirelessly transmitting information (e.g. a Bluetooth® transmitter). In this way, the circuit board can integrate with a remote processor (e.g. server, gateway, tablet, smartphone, computer, infusion pump, or some combination thereof) that can collectively analyze PVP waveforms and complementary information from the patch sensor.

The iPIVA sensor described herein simplifies traditional measurements of vital signs and hemodynamic parameters, which can involve multiple devices and can take several minutes to accomplish. The remote processor—which wirelessly couples with both the iPIVA sensor and patch sensor—can additionally integrate with existing hospital infrastructure and notification systems, such as a hospital electronic medical records (herein “EMR”) system. Such a system can alarm and alert caregivers to changes in a patient's condition, thereby allowing them to intervene.

The patch sensor measures vital signs such as HR, HRV, RR, SpO2, TEMP, and BP, along with complex hemodynamic parameters such as SV, CO, and FLUIDS. Measurement of BP is typically cuffless and calibrated with a cuff-based device, such as one based on oscillometry. The patch sensor is typically a body-worn device that adheres to a patient's chest and continuously and non-invasively measures the above-mentioned parameters. The chest is an ideal location when such measurements are made on hospital-based patients: it is usually easily accessible, and a sensor placed there is typically unobtrusive, comfortable, and removed from the hands (which typically undergo relatively large amounts of motion). Because the patch sensor is small and therefore considerably less noticeable and obtrusive than various other patient-monitoring devices, emotional discomfort over wearing it can be reduced, thereby fostering long-term compliance, healing, and general patient well-being.

Alternatively, in place of the patch sensor, the system providing independent measurements of HR, RR, and hemodynamic parameters can be a conventional vital sign or hemodynamic monitor, such as the Starling™ SV patient monitor manufactured by Cheetah Medical based in Newton Center, MA, USA.

The patch sensor can also include a motion-detecting accelerometer and gyroscope, from which it can determine motion-related parameters such as posture, degree of motion, activity level, respiratory-induced heaving of the chest, and falls. Such parameters could determine, for example, a patient's posture or movement during a hospital stay. The patch sensor can operate additional algorithms that process the motion-related parameters, allowing it to only measure vital signs and hemodynamic parameters when motion is minimized or below a predetermined threshold, thereby reducing artifacts. Moreover, the patch sensor estimates motion-related parameters such as posture to improve the accuracy of calculations for vital signs and hemodynamic parameters.

Disposable electrodes on a bottom surface of the patch sensor secure it to the patient's body without requiring bothersome cables. In embodiments, such electrodes easily connect to (and disconnect from) the sensor by means of magnets, thus allowing the sensor to easily snap back into proper position if it is removed. The patch sensor is typically lightweight, weighing about 20 grams. It is powered with a Li:ion battery that can be recharged with a conventional cable or using a wireless mechanism.

Given the above, in one aspect, the invention provides an IV system for monitoring a patient that is positioned on the patient's body. The IV system includes: 1) a catheter that inserts into the patient's venous system; 2) a pressure sensor connected to the catheter that measures physiological signals indicating a pressure in the patient's venous system; 3) a motion sensor that measures motion signals; and 4) a processing system that: i) receives the physiological signals from the pressure sensor; ii) receives the motion signals from the motion sensor; iii) processes the motion signals by comparing them to a pre-determined threshold value to determine when the patient has a relatively low degree of motion; and iv) process the physiological signals to determine a physiological parameter when the processing system determines that the motion signals are below the pre-determined threshold value.

In another aspect, the motion sensor is used to measure the patient's posture, as opposed to their motion, and the processing system determines the physiological parameter when the patient is in a pre-determined posture.

In another aspect, the invention provides an IV system for monitoring a patient that includes: 1) a catheter that inserts into the patient's venous system; 2) a pressure sensor connected to the catheter that measures physiological signals indicating a pressure in the patient's venous system; 3) a motion sensor that measures motion signals; and 4) a processing system that only transmits the physiological signals, or parameters calculated from these signals, when the motion signals fall below a pre-determined threshold.

In embodiments, the motion sensor is an accelerometer (e.g. a 3-axis accelerometer) and/or a gyroscope. In embodiments, the processing system calculates a motion vector by analyzing a motion signal corresponding to each axis of the 3-axis accelerometer. The pre-determined motion threshold used to determine if the patient's motion is too severe to make an accurate measurement typically corresponds to a vector magnitude of 0.1 G. In other embodiments, the processing system compares the motion vector to a pre-determined look-up table to determine the patient's posture.

In other embodiments, the processing system digitally filters the signals (e.g. with a digital high-pass filter) to generate a filtered signal. It then processes the filtered signal to determine the patient's heart/respiration rates. In embodiments, the processing system additionally processes the signal components indicating the patient's heart rate and respiration rate to determine a physiological parameter (e.g. wedge pressure, central venous pressure, blood volume, fluid volume, and pulmonary arterial pressure) indicating the patient's fluid status.

In embodiments, the processing system transforms the signals into the frequency domain to generate a frequency-domain signal prior to determining the physiological parameter. The method for the transform is typically an FFT, continuous wavelet transform, or a discrete wavelet transform.

In another aspect, the invention provides a system for monitoring a patient while simultaneously supplying IV fluids to the patient. The system features a housing positioned on the patient's body. The housing includes a catheter that inserts into the patient's venous system to supply the IV fluids, and a pressure sensor connected to the housing that measures time-dependent pressure signals indicating a pressure in the patient's venous system. The housing also includes a circuit system connected to the pressure sensor that receives the time-dependent signals it generates. The circuit system features: i) a differential amplifier that amplifies the time-dependent pressure signals to generate an amplified signal; ii) a low-pass filter that filters the amplified signal to generate a filtered signal, and iii) a secondary amplifier system that amplifies the filtered signal to generate a twice-amplified signal.

In embodiments, the differential amplifier, low-pass filter, and secondary amplifier can be positioned in any order within a circuit that differs from that described above.

In another aspect, the system additionally includes a processing system operating computer code that analyzes the twice-amplified signal to estimate a vital sign (e.g. HR, RR) corresponding to the patient. And in yet another aspect, the system additionally includes a wireless transmitter that transmits a digital representation of the vital sign to a remote receiver, and a power source that supplies power to the pressure sensor, circuit system, processing system, and wireless transmitter.

In embodiments, the IV system that includes a housing and completely encloses the circuit system and the pressure sensor, and attaches to the catheter. The catheter, for example, can be worn on the patient's hand or arm.

In embodiments, the differential amplifier features a gain of at least 10×. The low-pass filter typically separates out from the amplified signal a signal component containing heart rate and respiration rate components. The low-pass filter typically includes circuit components that generate a filter cutoff of between 10 and 30 Hz. In other embodiments, the circuit system additionally includes a high-pass filter that receives the twice-amplified signals and, in response, generates a twice-filtered signal. In this case, the high-pass filter typically includes circuit components that generate a filter cutoff of between 0.01 and 1 Hz.

In embodiments, the circuit system additionally includes a secondary low-pass filter that receives the twice-amplified signals and, in response, generates a thrice-filtered signal. In this case, the secondary low-pass filter typically includes circuit components that generate a filter cutoff of between 10 and 30 Hz.

In other embodiments, the circuit system additionally includes a motion sensor, such as an accelerometer or gyroscope. In other embodiments, the circuit system additionally includes a wireless transmitter, such as a Bluetooth®, Wi-Fi, or cellular transmitter. In other embodiments, the circuit system additionally includes a microprocessor that operates an algorithm to process the twice-amplified signal, or a signal derived therefrom. And in still other embodiments, the circuit system additionally includes a flash memory system that stores a digital representation of the twice-amplified signal or a signal derived therefrom.

In another aspect, the invention provides a system for monitoring a patient that includes a physiological sensor, connected to the patient, that features a bio-impedance and/or bio-reactance sensing element that measures a first set of parameters indicating the patient's fluid status. The system also includes an IV system featuring: 1) a catheter that inserts into the patient's venous system; 2) a pressure sensor that receives fluids from the catheter and, in response, measures a waveform indicating a pressure in the patient's venous system; and 3) a first processing system that receives the waveform and process it, or new signals derived from it, to estimate a second set of parameters indicating the patient's fluid status. A second processing system then receives the first and second sets of parameters, or a new parameter derived from them, and collectively process them to estimate a physiological parameter from the patient.

In another aspect, the invention provides a similar system, only the physiological sensor is worn on the patient. It includes the bio-impedance and/or bio-reactance sensing element and the first processing system.

In yet another aspect, the invention provides a system for monitoring a patient that includes: 1) a bio-impedance and/or bio-reactance sensing element connected to the patient that measures a first time-dependent waveform; 2) an IV system inserted in the patient's venous system featuring a pressure sensor that measures a second time-dependent waveform; and 3) a processing system that analyzes parameters calculated from both the first and second waveforms and collectively process them to estimate a physiological parameter from the patient.

In embodiments, the second processing system is selected from the group consisting of a computer, tablet computer, and mobile phone. This system can operate an algorithm that compares the first set of parameters to the second set of parameters to estimate the physiological parameter. In other embodiments, the physiological sensor includes a first wireless transmitter, the IV system includes a second wireless transmitter, and the second processing system includes a third wireless transmitter. Here, the third wireless transmitter can wirelessly communicate with both the first and second wireless transmitters.

In other embodiments, the first set of parameters indicating the patient's fluid status are selected from a group including BP, SpO2, SV, stroke index, CO, cardiac index, thoracic impedance, FLUIDS, inter-cellular fluids, and extra-cellular fluids. In other embodiments, the second set of parameters are selected from a group including F0, F1, energies associated with F0 and F1, mathematical combinations of F0 and F1, and parameters determined from these.

The second processing system can operate a linear mathematical model to collectively process the first and second sets of parameters. Alternatively, it can operate an algorithm based on artificial intelligence to collectively process the first and second sets of parameters.

In embodiments, the physiological parameter estimated by the second processing system indicates the patient's fluid status. For example, the physiological parameter estimated can be one of the patient's blood volume, wedge pressure, and pulmonary arterial pressure.

In another aspect, the invention provides a system for monitoring a patient that includes: 1) a physiological sensor connected to the patient and featuring sensing elements that measure a first set of signals indicating the patient's physiology; 2) an IV system featuring: i) a catheter that inserts into the patient's venous system; and ii) a pressure sensor that senses fluids from the catheter and, in response, measures a second set of signals indicating a pressure in the patient's venous system; and 3) a processing system that receives the first and second sets of signals and collectively process them, or new signals derived from them, to estimate a physiological parameter indicating the patient's status.

In another aspect, the invention provides a similar system, only all the elements—the physiological sensor, the pressure sensor, and the processing system—are worn on the patient's body.

And in yet another aspect, the invention provides a system for monitoring a patient that features: 1) a physiological sensor worn on the patient's body with sensing elements that measure heart rate and/or respiration rate; 2) a catheter that inserts into the patient's venous system and collects a fluid; 3) a pressure sensor connected to the catheter that senses the fluid and, in response, measures signals indicating a pressure in the patient's venous system; and 4) a processing system that receives the value of heart rate and/or respiration rate from the physiological sensor, and collectively process this value and the signals indicating the pressure in the patient's venous system, or new signals derived from these, to estimate a physiological parameter indicating the patient's status.

In embodiments, the physiological sensor measures an ECG waveform, and then process this to determine a value of HR. The physiological sensor can also measure an IPG or BR waveform, and then process this to determine a value of RR. In these embodiments, both HR and RR represent the ‘first set of signals’, as used herein.

In embodiments, the pressure sensor measures a time-dependent pressure waveform indicating pressure in the patient's venous system; this represents the ‘second set of signals’, as used herein. The processing system can then be configured to process the time-dependent waveform with an algorithm (e.g., an algorithm for performing an FFT, continuous wavelet transform, or discrete wavelet transform) to generate a frequency-domain spectrum. In one embodiment, the processing system then collectively processes the value of HR and the frequency-domain spectrum to determine a feature in the frequency-domain spectrum corresponding to HR (i.e. F1); it then processes F1 or a parameter estimated therefrom (e.g. its amplitude or corresponding energy, as described herein) to estimate the physiological parameter indicating the patient's status. In a related embodiment, the processing system collectively processes the value of RR and the frequency-domain spectrum to determine a feature in the frequency-domain spectrum corresponding to RR (i.e. F0); it then processes F0 or a parameter estimated therefrom (e.g. its amplitude or corresponding energy, as described herein) to estimate the physiological parameter indicating the patient's status. In yet another embodiment, both F0 and F1, or parameters derived therefrom, are collectively processed to estimate the physiological parameter indicating the patient's status. This parameter can be, e.g., wedge pressure, central venous pressure, pulmonary arterial pressure, blood volume, fluid volume, or a related value.

In another aspect, the invention provides an IV system for monitoring a patient that is positioned on the patient's body. The system features: 1) a catheter that inserts into the patient's venous system; 2) a pressure sensor connected to the catheter that measures signals indicating a pressure in the patient's venous system; and, 3) a processing system that receives the signals from the pressure sensor and, in response, process them to measure a physiological parameter.

In another aspect, the invention provides an IV system for monitoring a patient that is positioned on the patient's body. The system features: 1) a catheter that inserts into the patient's venous system; 2) a pressure sensor connected to the catheter that measure signals indicating a pressure in the patient's venous system; and, 3) a processing system that receives the signals from the pressure sensor and process them to determine signal components indicating either (or both) of the patient's heart rate and respiration rate.

In yet another aspect, the invention provides a system for monitoring a patient that is positioned on the patient's body. The system features: 1) a catheter that inserts into the patient's venous system and collects a fluid; 2) a pressure sensor connected to the catheter that senses the fluid and, in response, measure signals indicating a pressure in the patient's venous system; and, 3) a processing system that receives the signals from the pressure sensor and, in response, processes them to determine either (or both) of the patient's heart rate and respiration rate.

In embodiments, the processing system digitally filters the signals (e.g. with a digital high-pass filter, low-pass filter, and/or band-pass filter) to generate a filtered signal. It then processes the filtered signal to determine the patient's heart/respiration rate. In embodiments, the processing system additionally processes the signal components indicating the patient's heart rate and respiration rate to determine a physiological parameter (e.g. F0, F1, energy associated with F0, energy associated with F1, wedge pressure, central venous pressure, blood volume, fluid volume, and pulmonary arterial pressure) indicating the patient's fluid status.

In embodiments, the processing system transforms the signals into the frequency domain to generate a frequency-domain signal. The method for the transform is typically an FFT, continuous wavelet transform (herein “CWT”), or a discrete wavelet transform (herein “DWT”).

In embodiments, the processing system is a microprocessor. The microprocessor typically includes a random-access memory that stores a computer program, and a flash memory that stores a digital representation of the signals from the pressure sensor. In other still other embodiments, the processing system additionally includes a motion sensor, such as an accelerometer or gyroscope. In other embodiments, the processing system additionally includes a wireless transmitter, such as a Bluetooth®, Wi-Fi, or cellular transmitter.

In another aspect, the invention provides an IV system that monitors a patient and is positioned in its entirety on the patient's body. The IV system includes: 1) a catheter that inserts into the patient's venous system; 2) a pressure sensor connected to the catheter that measures signals indicating pressure in the patient's venous system; and, 3) a circuit system that receives the signals from the pressure sensor. The circuit system features: i) a differential amplifier that amplifies the signals to generate an amplified signal; ii) a low-pass filter that filters the amplified signal to generate a filtered signal; and iii) a secondary amplifier system that amplifies the filtered signal to generate a twice-amplified signal.

In another aspect, the invention provides a similar IV system, also positioned in its entirety on the patient's body, that includes a catheter, pressure sensor, and circuit system similar to those described above. Here, the circuit system features: i) an amplifier that amplifies the signals to generate an amplified signal; ii) a filter that filters the amplified signal to generate a filtered signal; iii) a secondary amplifier system that amplifies the filtered signal to generate a twice-amplified signal; and iv) an analog-to-digital converter that digitizes the twice-amplified signal, or a signal derived therefrom.

In embodiments, the amplifiers, filters, and secondary filters described above can be arranged in any order within the circuit system.

In yet another aspect, the invention provides a system for monitoring a patient featuring a catheter that inserts into the patient's venous system, and a housing positioned in its entirety on the patient's body that encloses: 1) a pressure sensor configured to sense fluids from the catheter and, in response, measure pressure signals; and 2) a circuit system with circuit elements that amplify, filter, and digitize the pressure signals to identify the signal components indicating the patient's HR and RR.

In embodiments, the IV system includes a housing that completely encloses the circuit system and the pressure sensor, and attaches to the catheter. The housing, for example, can be worn on the patient's hand or arm. For example, it can be attached to these body parts using a band or adhesive.

In embodiments, the differential amplifier features a gain of at least 10×. The low-pass filter typically separates the amplified signal into a first amplified signal component containing components related to both HR and RR, and a second amplified signal component that lacks these components. The low-pass filter typically includes circuit components that generate a filter cutoff of between 10 and 30 Hz. In other embodiments, the circuit system additionally includes a high-pass filter that receives the twice-amplified signals and, in response, generates a twice-filtered signal. In this case, the high-pass filter typically includes circuit components that generate a filter cutoff of between 0.01 and 1 Hz.

In embodiments, the circuit system additionally includes a secondary low-pass filter that receives the twice-amplified signals and, in response, generates a thrice-filtered signal. In this case, the secondary low-pass filter typically includes circuit components that generate a filter cutoff of between 10 and 30 Hz.

In other embodiments, the circuit system additionally includes a motion sensor, such as an accelerometer or gyroscope. In other embodiments, the circuit system additionally includes a wireless transmitter, such as a Bluetooth®, Wi-Fi, or cellular transmitter. In other embodiments, the circuit system additionally includes a microprocessor that operates an algorithm to process the twice-amplified signal, or a signal derived therefrom. And in still other embodiments, the circuit system additionally includes a flash memory system that stores a digital representation of the twice-amplified signal or a signal derived therefrom.

Advantages of the invention should be apparent from the following detailed description, and from the claims.

### BRIEF DESCRIPTION OF THE DRAWINGS

FIG. 1 is a drawing of the system of the invention featuring both a patch sensor and an iPIVA sensor;

FIG. 2A is a schematic drawing indicating how the iPIVA sensor of FIG. 1 attaches to a patient;

FIG. 2B is a mechanical drawing of an arm-worn housing that encloses a circuit board used in the iPIVA sensor;

FIG. 2C is an image of the circuit board enclosed by the arm-worn housing shown in FIG. 2B;

FIG. 2D is a photograph of the circuit board indicated by the image shown in FIG. 2C;

FIG. 3 is an electrical schematic of a circuit board of FIGS. 2D and 2E featuring circuits for filtering, amplifying, and digitizing PVP-AC and PVP-DC waveforms;

FIG. 4A is a time-dependent plot of a first PVP-AC waveform measured after a first amplifier stage described by the electrical schematic of FIG. 3;

FIG. 4B is a time-dependent plot of a second PVP-AC waveform measured after a second amplifier/filter stage described by the electrical schematic of FIG. 3;

FIG. 4C is an electrical schematic of a circuit board, taken from the electrical schematic of FIG. 3, featuring a circuit for processing PVP-AC waveforms;

FIG. 5 is a logarithmic, frequency-dependent plot of PVP-AC and PVP-DC signals measured with the circuit board of FIG. 2E compared to the theoretical, ideal responses of filters and amplifiers described by the electrical schematics of FIG. 3 and fabricated on the circuit board of FIG. 2E;

FIG. 6A is a time-dependent plot of a PVP-AC waveform measured from a patient over a 30-minute period with the system according to the invention;

FIGS. 6B, 6C, and 6D are time-dependent plots of PVP-AC waveforms (i.e. waveform snippets) taken from the plot in FIG. 6A and beginning at time periods of, respectively, 420, 780, and 1310 seconds;

FIGS. 6E, 6F, and 6G are frequency-domain spectra representing the FFTs of the waveform snippets shown, respectively, in FIGS. 6B, 6C, and 6D;

FIG. 7 is a mechanical drawing of a iPIVA physiological sensor of FIG. 1;

FIGS. 8A-8E are time-dependent plots of ECG, PPG, IPG/BR, PCG, and PVP-AC waveforms measured simultaneously by the patch sensor and iPIVA sensor of FIG. 1;

FIGS. 9A, 9B, and 9C are mechanical drawings of, respectively, a bottom surface, top surface, and exploded view of a iPIVA physiological sensor according to the invention;

FIGS. 10A, 10B, and 10C are, respectively, a schematic drawing of a patient wearing an embodiment of an iPIVA physiological sensor according to the invention, a time-dependent plot of a PPG waveform measured with the iPIVA physiological sensor of FIG. 10A, and a time-dependent plot of a PVP-AC waveform measured with the iPIVA physiological sensor of FIG. 10A;

FIGS. 11A, 11B, and 11C are, respectively, a schematic drawing of a patient wearing an embodiment of an iPIVA physiological sensor according to the invention, a time-dependent plot of a PPG waveform measured with the iPIVA physiological sensor of FIG. 11A, and a time-dependent plot of a PVP-AC waveform measured with the iPIVA physiological sensor of FIG. 11A;

FIGS. 12A, 12B, 12C, and 12D are, respectively, a schematic drawing of a patient wearing an embodiment of an iPIVA physiological sensor according to the invention, a time-dependent plot of a PPG waveform measured with the iPIVA physiological sensor of FIG. 12A, a time-dependent plot of a PCG waveform measured with the iPIVA physiological sensor of FIG. 12A, and a time-dependent plot of a PVP-AC waveform measured with the iPIVA physiological sensor of FIG. 12A;

FIGS. 13A, 13B, 13C, 13D, and 13E are, respectively, a schematic drawing of a patient wearing an embodiment of an iPIVA physiological sensor according to the invention, a time-dependent plot of an ECG waveform measured with the iPIVA physiological sensor of FIG. 13A, a time-dependent plot of a PPG waveform measured with the iPIVA physiological sensor of FIG. 13A, a time-dependent plot of an IPG/BR waveform measured with the iPIVA physiological sensor of FIG. 13A, and a time-dependent plot of a PVP-AC waveform measured with the iPIVA physiological sensor of FIG. 13A;

FIGS. 14A, 14B, 14C, 14D, 14E, and 14F are, respectively, a schematic drawing of a patient wearing an embodiment of an iPIVA physiological sensor according to the invention, a time-dependent plot of an ECG waveform measured with the iPIVA physiological sensor of FIG. 14A, a time-dependent plot of a PPG waveform measured with the iPIVA physiological sensor of FIG. 14A, a time-dependent plot of an IPG/BR waveform measured with the iPIVA physiological sensor of FIG. 14A, a time-dependent plot of a PCG waveform measured with the iPIVA physiological sensor of FIG. 14A, and a time-dependent plot of a PVP-AC waveform measured with the iPIVA physiological sensor of FIG. 14A;

FIG. 15A is a flow chart showing an algorithm used by the system in FIG. 1 that processes signals from both the iPIVA and patch sensors to monitor a patient;

FIG. 15B is a time-dependent plot of, respectively, the ECG, PPG, and IPG/BR waveforms shown in FIGS. 8A, 8B, and 8C;

FIG. 15C is a time-dependent plot of a PVP-AC waveform (referred to in the flow chart of FIG. 15A as ‘PVP-ACₜᵢₘₑ’) measured with the iPIVA sensor;

FIG. 15D is a time-dependent plot of a waveform snippet (referred to in the flow chart of FIG. 15A as ‘PVP-AC_{time, segment}’) taken from the time-dependent plot of the PVP-AC waveform in FIG. 15C; and,

FIG. 15E is a frequency-domain spectrum (referred to as ‘PVP-AC_{frequency, segment, ave}’) showing an ensemble average of DWTs of the time-domain waveform snippets indicated in FIG. 15C.

### DETAILED DESCRIPTION

Although the following text sets forth a detailed description of numerous different embodiments, it should be understood that the legal scope of the invention described herein is defined by the words of the claims set forth at the end of this patent. The detailed description is to be construed as exemplary only; it does not describe every possible embodiment, as this would be impractical, if not impossible. One of ordinary skill in the art could implement numerous alternate embodiments, which would still fall within the scope of the claims.

### iPIVA Sensor

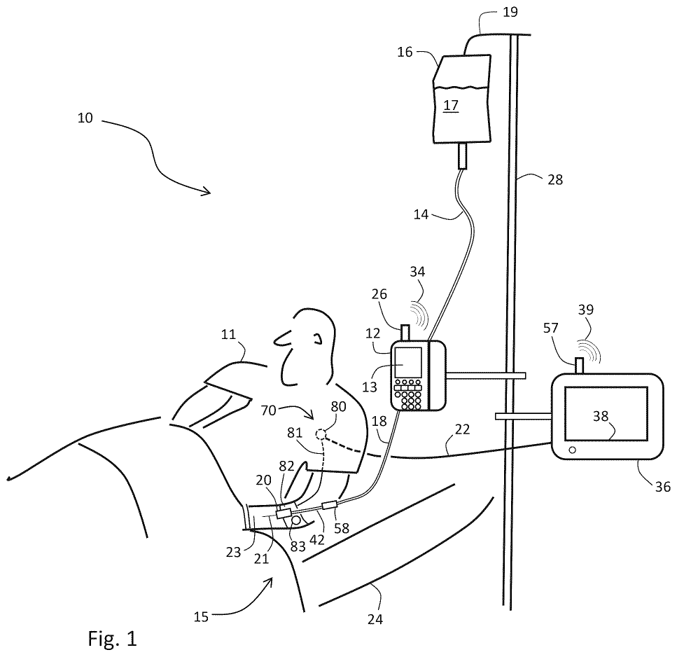

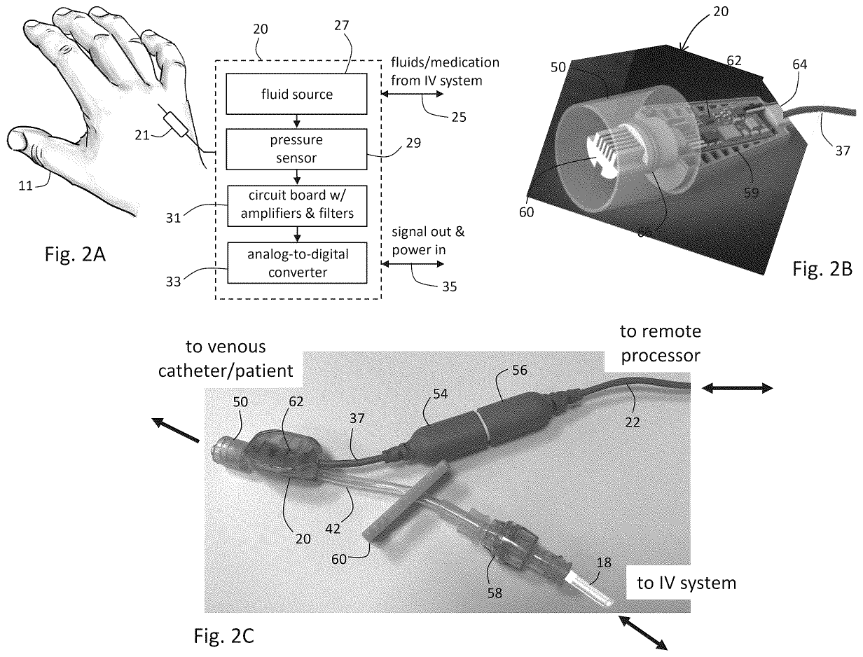

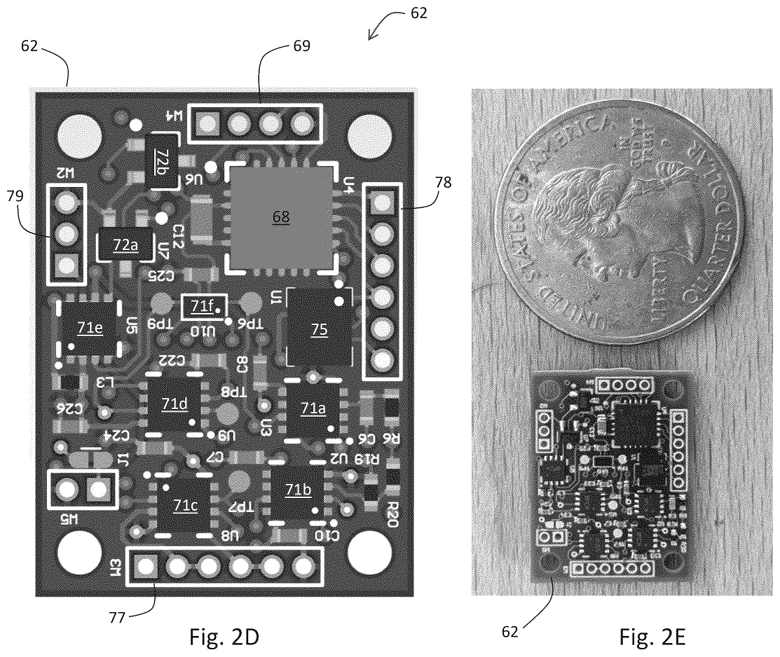

Referring to FIG. 1, a system 10 featuring an IV system 19 incorporating an iPIVA sensor 15, working in concert with a iPIVA physiological sensor 70, characterizes vital signs and hemodynamic parameters from a patient 11 deposed in a hospital bed 24. The iPIVA sensor 15 includes an arm-worn housing 20 that encloses a fiberglass circuit board (shown in FIGS. 2B and 2D, and described in detail below) configured to amplify, filter, and digitize PVP signals. The arm-worn housing 20 terminates with a venous catheter 21 inserted into a vein in the patient's hand or arm. A remote processor 36 (e.g. a tablet computer or device with comparable functionality) connects to the arm-worn housing 20 through a cable 22, and to the iPIVA physiological sensor 70 through a wireless interface (e.g. Bluetooth®). In embodiments, the remote processor 36 can connect to both the arm-worn housing 20 and iPIVA physiological sensor 70 through wired (e.g. cable) or wireless (e.g. Bluetooth®) means. During a measurement, it receives and PVP signals from the iPIVA sensor 15 and vital signs and hemodynamic parameters from the iPIVA physiological sensor 70, and collectively analyzes them as described in detail below to monitor the patient.

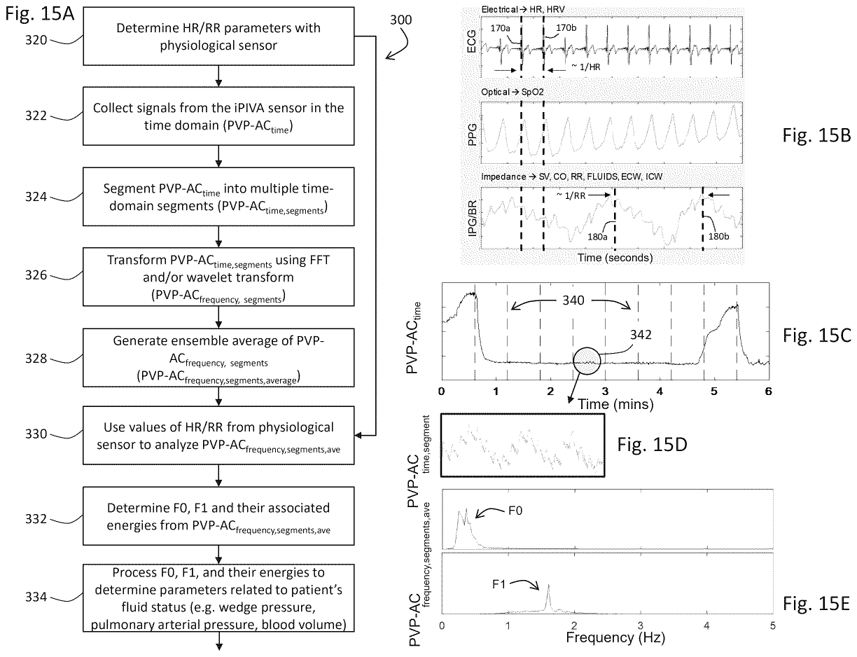

Both the iPIVA sensor 15 and iPIVA physiological sensor 70 are tightly coupled and integrated within the IV system 19. It is the combination of these components, along with the collective analysis of the information they measure (e.g. by the remote processor), that is the focus of the invention described herein. More specifically, during a measurement, the iPIVA physiological sensor 70 measures the patient's vital signs (e.g. HR, HRV, RR, BP, SpO2, TEMP) and hemodynamic parameters (SV, CO, FLUIDS), while the iPIVA sensor 15 measures PVP waveforms that, with processing, yield F0 and F1. Digital versions of these data sets flow to the remote processor 36 for follow-on processing. For example, in embodiments, the remote processor 36 analyzes the digitized PVP waveforms and calculates their frequency-domain transform—using techniques such as FFTs, CWTs, and DWTs—to yield a frequency-domain spectrum. It then uses HR and RR values from the iPIVA physiological sensor 70 to detect F0 and F1 from the frequency-domain spectrum, and then determines the associated energies of these features, to estimate a parameter indicating a patient's fluid status (e.g. wedge pressure). In embodiments, energies associated with F0 and F1, along with measurements from the iPIVA physiological sensor, can be used to estimate other parameters related to the patient's fluid status, such as pulmonary arterial pressure and blood volume, as described in more detail below with reference to FIG. 15A. The remote processor can also include an internal wireless transmitter (e.g. a Bluetooth® or Wi-Fi transmitter) that sends information through an antenna 57 to the hospital's EMR system, as indicated by the icon 39. It can also generate audio and/or visual ‘alarms’and ‘alerts’when physiological parameters measured by the iPIVA sensor 15 and iPIVA physiological sensor 70 indicate the patient's status trend above or below certain pre-determined thresholds, thereby indicating the patient is decompensating.

The IV system 19 features a bag 16 containing pharmaceutical compounds and/or fluid (herein “medication” 17) for the patient. The bag 16 connects to an infusion pump 12 through a first tube 14. A standard IV pole 28 supports the bag 16, the infusion pump 12, and the remote processor 36. A display 13 on the front panel of the infusion pump 12 indicates the type of medication delivered to the patient, its flow rate, measurement time, etc. Medication 17 passes from the bag 16 through the first tube 14 and into the infusion pump 12. From there, it is metered out appropriately, and passes through a second tube 18, through a connector 58 and cable segment 42, into the arm-worn housing 20, and finally through the venous catheter 21 and into the patient's venous system 23. The arm-worn housing 20 is typically affixed to the patient's arm or hand, e.g. using an adhesive such as medical tape or a disposable electrode.

The venous catheter 21 may be a standard venous access device, and thus may include a needle, catheter, cannula, or other means of establishing a fluid connection between the catheter 21 and the patient's peripheral venous system 23. The venous access device may be a separate component connected to the venous catheter 21, or may be formed as an integral portion of it. In this way, the IV system 19 supplies the medication 17 to the patient's venous system 23 while the iPIVA sensor 15 and iPIVA physiological sensor 70, which features a pressure-measuring system and described in more detailed below, simultaneously measures signals related to the patient's PVP, vital signs, and hemodynamic parameters.

Importantly, and as described in more detail below, the arm-worn housing 20 is designed so that it is in constant ‘fluid connection’ with the patient's circulatory system (and particularly the venous system) while being deployed close to (or directly on) the patient's body. It features electronic systems for measuring analog pressure signals within the patient's venous system to generate PVP waveforms, and then amplifying and filtering these to optimize their signal-to-noise ratios. An analog-to-digital converter within the arm-worn housing digitizes the analog PVP waveforms prior to transmitting them through the cable, thereby minimizing any noise (caused, e.g., by the cable's motion) that would normally affect transmitted analog signals and ultimately introduce inaccuracies into values of F0 and F1 (and their associated energies) measured downstream. Notably, this design provides a relatively short conduction path between where the PVP waveforms are first detected and then processed and digitized; ultimately this results in signals that are more likely to yield highly accurate values of wedge pressure (and in embodiments pulmonary arterial pressure (and particularly the diastolic component on this pressure), blood volume and other fluid-related parameters).

FIGS. 2A-D show in more detail the arm-worn housing 20, its method of operation, and various component included therein. The housing 20 is designed to rest comfortably close to or on the patient while: 1) allowing fluids (and/or medication) from the IV system to flow (as indicated by arrow 25 in FIG. 2A) into the patient's venous system (box 27 in FIG. 2A); 2) measuring pressure signals from the patient's venous system with a pressure sensor (box 29 in FIG. 2A); 3) filtering/amplifing the pressure signals with a small-scale printed circuit board featuring circuits functioning as analog amplifiers and filters (box 31 in FIG. 2A); 4) digitizing the filtered/amplified signals with an analog-to-digital converter (box 33 in FIG. 2A); and 5) transmitting the digitized signals using a serial protocol (e.g. SPI, I2C) for further processing by the remote processor (arrow 35 in FIG. 2A).

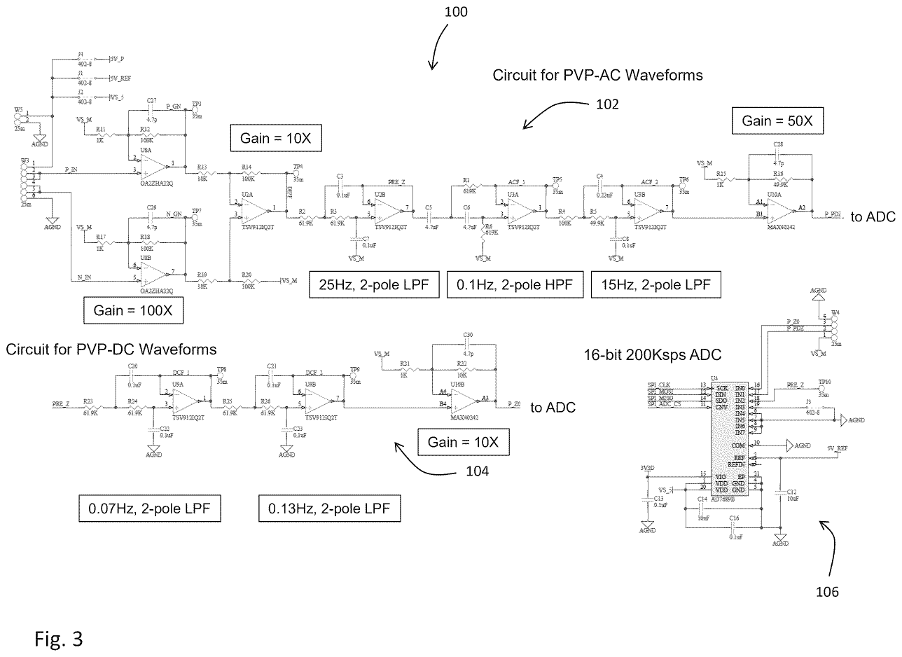

FIGS. 2B and 2C show, respectively, a mechanical drawing of the arm-worn housing 20 enclosing the circuit board 62 according to the invention, and a photograph of the arm-worn housing 20 connected to the second tube 18 (which receives medication from the IV system) and the cable 22 (which transmits signals to the remote processor). Specifically, the circuit board 62 supports a collection of integrated circuits (herein “ICs”) and discrete electrical components that, while working in concert, perform the functions shown schematically in FIG. 2A; they are deployed on the circuit board 62 according to an electrical schematic shown in FIG. 3 and described in more detail below. The circuit board 62 connects through a back panel 64 on the housing's distal end to a short cable segment 37 terminated with a multi-pin connector (not shown in the figure) and enclosed by an overmold 54 that mates with a corresponding connector (also not shown in the figure) enclosed by a similar overmold 56. The overmold 56 connects to the cable 22, which in turn connects to the remote processor 36. With this mechanism, the cable 22 can be easily detached from the arm-worn housing 20, e.g. in case the patient is moved or connected to a new infusion system. The cable 22 features individual electrical connectors that supply power (5V, 3.3V, GND) to the circuit board, and additionally transmit digitized PVP waveforms over a serial protocol (e.g. SPI, I2C) to the remote processor 36 for follow-on processing, as is described in more detail below. In other embodiments, the circuit board 62 can include an internal wireless transceiver (e.g. Bluetooth® Wi-Fi, or cellular transceiver) so that it can wirelessly communicate with remote systems, such as the remote processor, infusion pump, and the hospital's EMR. It may also include an accelerometer to estimate motion of the arm-worn housing 20, flash and RAM memory to store information, a high-end microprocessor for analyzing PVP waveforms and other signals, a battery, and additional circuitry and sensors for measuring TEMP and physiological waveforms (e.g. PPG, ECG, IPG, and BR) from which vital signs (PR, HR, HRV, SpO2, RR, BP) and hemodynamic parameters (FLUID, SV, CO) are calculated. In general, the circuit board 62 is designed to amplify and condition PVP signals along with other physiological signals with an approach comparable to that deployed in conventional vital sign monitors, such as those described in U.S. Pat. Nos. 10,314,496 and 10,188,349, the contents of which are incorporated herein by reference.

Referring to FIG. 2B, the arm-worn housing 20 features a connector 60 surrounded by a flange 50 that connects to an in-dwelling venous catheter (not shown in the figure) which, during a measurement, inserts into the patient's venous system. The catheter is typically housed in a mated plastic component (also not shown in the figure) that secures to the flange 50 and forms a waterproof seal using a rubber gasket 66. The circuit board 62 is held securely in place within the arm-worn housing with a set of plastic ribs 59 It connects to the cable 22 with the short cable segment 37 that is typically just a few centimeters in length.

FIGS. 2D and 2E show, respectively, an image and photograph of the circuit board 62 within the arm-worn housing. The circuit board 62 was fabricated according to an electrical schematic, shown in FIG. 3 (specifically component 100) and described in more detail below. The circuit board 62 shown in the figure is a 4-layer fiberglass/metal structure that includes metal pads soldered to, among other components, an analog-to-digital converter 68, accelerometer 75, operational amplifiers 71a-f, and power regulators 72a-b. More specifically, operational amplifiers 71a-d make up analog high and low-pass filters, and operational amplifiers 71e-f and power regulators 72a-b collectively regulate power levels for the various components in the circuit board 62. The accelerometer 75 measures motion of the circuit board 62 and, in doing this, any part of the patient's body it is attached to. The analog-to-digital converter 68 digitizes analog PVP waveforms after they have been filtered, and converts them into digital waveforms with 16-bit resolution and a maximum digitization rate of 200 Ksamples/second (herein “Ksps”).

The circuit board 62 additionally includes sets of metal-plated holes that support a 4-pin connector 69, two 6-pin connectors 77, 78, and a 3-pin connector 79. More specifically, connector 69 connects directly to the pressure transducer, where it receives a common ground signal and analog PVP waveforms representing pressure in the patient's venous system. These waveforms are filtered and digitized as described in more detail, below. Through the connector 79 the circuit board receives power (+5V, +3.3V, and ground) from an external power supply, e.g. a battery or power supply located in the arm-worn housing. These power levels may be different in other embodiments of the invention. Digital signals and a corresponding ground from the analog-to-digital converter 68 are terminated at connector 78; they leave the circuit board 62 at this point, e.g. through cable segment 37 shown in FIG. 2C. Connector 77 is used primarily for testing and debugging purposes, and in particular allows analog PVP signals, once they pass through analog high and low-pass filters, to be measured with an external device such as an oscilloscope.

In embodiments, the circuit board 62 additionally includes components for processing, storing, and transmitting data that are digitized by the analog-to-digital converter 68. For example, the circuit board 62 can include a microprocessor, microcontroller, or similar integrated circuit, and can additionally provide analog and digital circuitry for the iPIVA physiological sensor. In embodiments, the microprocessor or microcontroller thereon can operate computer code to process PVP-AC, PVP-DC, ECG, PCG, PPG, IPG, BP, and other time-dependent waveforms from both the iPIVA sensor and iPIVA physiological sensor to determine vital signs (e.g. HR, HRV, RR, BP, SpO2, TEMP), hemodynamic parameters (CO, SV, FLUIDS), components of PVP waveforms (e.g. F0, F1, and amplitudes and energies associated thereto), and associated parameters (e.g. wedge pressure, central venous pressure, blood volume, fluid volume, and pulmonary arterial pressure) related to the patient's fluid status. “Processing” by the microprocessor in this way, as used herein, means using computer code or a comparable approach to digitally filter (e.g. with a high-pass, low-pass, and/or band-pass filter), transform (e.g. using FFT, CWTs, and/or DWTs), mathematically manipulate, and generally process and analyze the waveforms and parameters and constructs derived therefrom with algorithms known in the art. Examples of such algorithms include those described in the following co-pending and issued patents, the contents of which are incorporated herein by reference: “NECK-WORN PHYSIOLOGICAL MONITOR”, U.S. Ser. No. 14/975,646, filed Dec. 18, 2015; “NECKLACE-SHAPED PHYSIOLOGICAL MONITOR”, U.S. Ser. No. 14/184,616, filed Aug. 21, 2014; and “BODY-WORN SENSOR FOR CHARACTERIZING PATIENTS WITH HEART FAILURE”, U.S. Ser. No. 14/145,253, filed Jul. 3, 2014.

In related embodiments, the circuit board can include both flash memory and random access memory for storing time-dependent waveforms and numerical values, either before or after processing by the microprocessor. In still other embodiments, the circuit board can include Bluetooth® and/or Wi-Fi transceivers for both transmitting and receiving information.

Referring again to FIG. 1 and FIGS. 2A-2E, during a measurement with the iPIVA sensor, the venous catheter delivers medication 17 metered out by the infusion pump 12, through the second tube 18, and into the patient's venous system 23. The second tube 18 is terminated with a connector 58 that connects to the arm-worn housing through a short cable segment 42. This allows the arm-worn housing to be easily decoupled (i.e. separated) from the IV system 19. In this embodiment, the second tube 18 can be temporarily pinched with a small plastic part 60 to occlude flow of fluid into and out of the patient. In related embodiments, the arm-worn housing 20 can include a power source (such as an internal battery), processor, and an on-board wireless transmitter. In this way, the iPIVA sensor 15 can function as a body-worn device for e.g. an ambulatory patient: it can measure PVP waveforms, processes them to determine energies associated with F0 and F1, and then transmits digitized versions of these components to a remote device. Such a system could also effectively couple with the iPIVA physiological sensor 70, which is also a body-worn vital sign and hemodynamic monitor that is both wireless and battery-powered, and can thus measure vital signs and hemodynamic parameters from the ambulatory patient. This means that, working in concert according to the above-mentioned embodiment, the iPIVA sensor and iPIVA physiological sensors can function as an effective, singular device for patients relegated to hospital beds, as well as those transferring to different areas of the hospital, and ultimately transitioning from the hospital to the home.

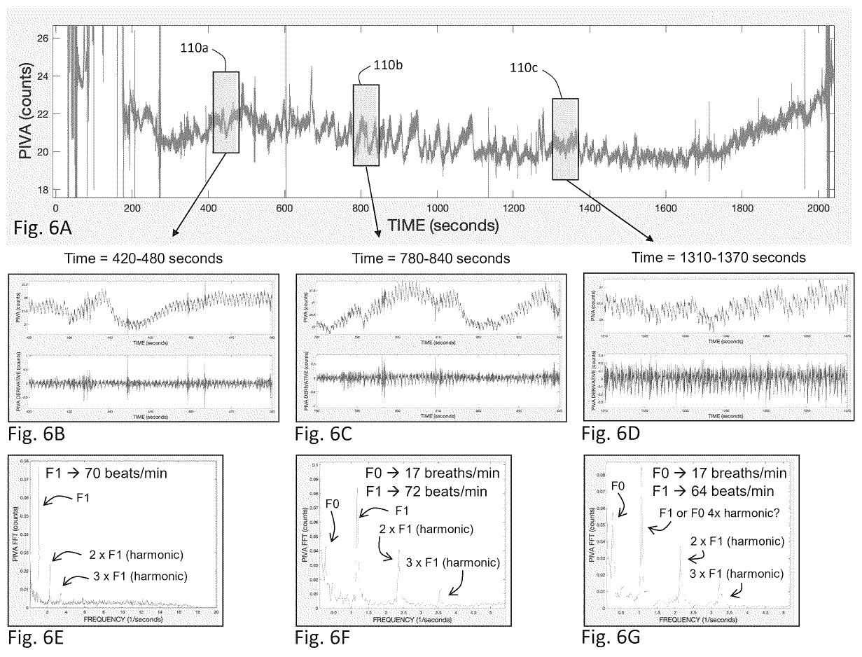

PVP waveforms measured with the system described herein feature signal components that relate to heartbeat and respiratory events that may vary rapidly with time. Such signal components are referred to herein as ‘PVP-AC’ waveforms, where ‘AC’ is a term normally used to describe alternating current, but is used herein to describe a signal component that changes rapidly in time as the signal evolves. FIGS. 6A-D show examples of PVP-AC waveforms, and how they are amplified and conditioned by the circuit board 62 in the arm-worn housing 20 to improve their signal-to-noise ratio. Likewise, low-frequency components of the PVP waveforms that are relatively stable and unvarying over time are referred to herein as “PVP-DC” waveforms, where the term ‘DC’ is normally used to describe direct current, but is used herein to describe signals that do not rapidly change with time.

More specifically, PVP waveforms typically have signal levels in the 5-50 V range, a relatively weak amplitude that can be difficult to process. Such signals have been described previously (e.g. in U.S. patent application Ser. No. 16/023,945 (filed Jun. 29, 2018 and published as U.S. Patent Publication 2019/0000326); U.S. patent application Ser. No. 14/853,504 (filed Sep. 14, 2015 and published as U.S. Patent Publication No. 2016/0073959), and PCT Application No. PCT/US 16/16420 (filed Feb. 3, 2016, and published as WO 2016/126856)). The contents of these pending patent applications have been previously incorporated herein by reference. In a conventional PIVA measurement, as described in these documents, PVP waveforms are measured with a pressure sensor proximal to the patient that generates analog signals; these typically pass through a relatively long cable, and are amplified, filtered, and digitized with a system located remotely from the patient. Additionally, conventional PIVA sensors, such as those previously disclosed, typically include transformation of the PVP waveforms into the frequency domain (typically using, e.g., a FFT), and then attempt to identify F0 (indicating a frequency related to RR) and F1 (indicating a frequency related to HR) without any secondary determination of these parameters. Energies associated with F0 and F1 are then analyzed to estimate other metrics (e.g. wedge pressure, pulmonary arterial pressure) related to the patient's fluid status. However, because PVP waveforms are so weak and characterized by low signal-to-noise ratios, they can be extremely difficult to measure. Additionally, when transformed into the frequency domain, signal components related to F0, F1, and their respective harmonics (i.e. frequencies corresponding to integer multiples of F0 and F1) may overlap with one another, making them difficult to delineate and explicitly measure. These and other factors may ultimately complicate the determination of parameters determined from energies associated with F0 and F1, e.g. the patient's fluid status.

The current invention attempts to cure these deficiencies in measuring PVP waveforms, and ultimately the energies associated with F0 and F1, by: 1) amplifying, filtering, digitizing, and in some cases processing PVP waveforms immediately after they are sensed by the pressure transducer (as opposed to first passing analog signals through a long, noise-inducing cable) to improve their signal-to-noise ratio and create a digital representation of them that is immune to cable-induced noise; 2) simultaneously and independently measuring HR and RR with an external iPIVA physiological sensor, which is tightly integrated with the iPIVA sensor; and 3) collectively processing the amplified/filtered/digitized PVP waveforms with HR and RR measurements from the iPIVA physiological sensor to better determine the energies associated with F0 and F1. Additionally, other measurements from the iPIVA physiological sensor, such as BP, SV, CO, and FLUIDS, and be combined with measurements from the iPIVA sensor to better determine the patient's fluid status, thereby improving their care within a hospital.

FIG. 3 shows a schematic 100 of the circuit board 62 described in FIGS. 2A-C. The schematic 100 includes: 1) a first set of circuit elements 102 designed to amplify and filter PVP-AC waveforms; 2) a second set of circuit elements 104 designed to amplify and filter PVP-DC waveforms; and 3) a 16-bit, 200 Ksps analog-to-digital converter 106 to digitize both the PVP-AC and PVP-DC waveforms.

More specifically, the circuit described by the schematic 100 is designed to serially perform the following function on incoming PVP waveforms:

### Incoming PVP Waveforms

1) Amplify the signal with 100× gain using a zero-drift amplifier
2) Differentially amplify the signal with an additional 10× gain
3) Filter the amplified signals with a 25 Hz, 2-pole low-pass filter

This first portion of the circuit provides roughly 1000× combined gain for the incoming PVP waveforms, thereby amplifying the input signal (which is typically in the V range) to a larger signal (in the mV range). The follow-on low-pass filter removes any high-frequency noise. Ultimately these steps facilitate processing of both the PVP-AC and PVP-DC waveforms, as described below.

In the descriptions provided herein, the term ‘differentially amplify’ refers to a process wherein the circuit measures the difference between positive (P_IN in FIG. 3) and negative (N_IN in FIG. 3) terminals. Notably, the output of the differential amplifier is a single-ended signal, zeroed at the midpoint voltage of the system. Alternatively, it could be zeroed at 0 V, although a centering point between the voltage rails generally provides a more accurate and cleaner output signal.

Likewise, the term ‘zero-drift amplifier’ refers to an amplifier that: 1) internally corrects for temperature and other forms of low-frequency signal error; 2) has very high input impedance; and 3) has very low offset voltages. The incoming signal received by a zero-drift amplifier is typically extremely small, meaning it can be subject to interference, gain shifts, or the amplifier inputs bleeding out generated current; the zero-drift architecture of the amplifier helps reduce or eliminate this.

After processing the input PVP waveforms, the circuit described by the schematic 100 is designed to serially perform the following function on PVP-AC and PVP-DC waveforms:

PVP-AC Waveforms Only
1) Filter the signal with a 0.1 Hz, 2-pole high-pass filter
2) Filter the signal with a 15 Hz, 2-pole low-pass filter
3) Amplify the signal with 50× gain

### PVP-DC Signal Only

1) Filter the signal with a 0.07 Hz, 2-pole low-pass filter
2) Filter the signal with a 0.13 Hz, 2-pole low-pass filter
3) Amplify the signal with 10× gain
   Both PVP-AC and PVP-DC waveforms 1) Digitize the signals with a 16-bit, 200 Ksps Delta-Sigma analog-to-digital converter

With this level of digital signal processing, the circuit board 62 can process PVP waveforms directly on the patient's body, and more specifically signals associated with respiration rate (F0) and heart rate (F1). It performs these functions without having to send signals through an external cable, which is an approach that can add noise and other signal artifacts and thus negatively impact measurement of F0, F1, and their associated energies as described above.

As appreciated by those skilled in the art, the circuit elements 102, 104, and 106 shown in FIG. 3 may have a comparable design that accomplishes the above-described steps with a schematic that differs slightly from that shown in FIG. 3. Additionally, it may include other integrated circuits and components to improve the measurement of F0, F1, and their associated energies, and thus provide added functionality. For example, the circuit board 62 may also include a temperature/humidity sensor, multi-axis accelerometer, integrated gyroscope, or other motion-detecting sensors configured to sense a motion signal associated with the patient (e.g. movement of the patient's arm, wrist, or hand). In embodiments, for example, the motion signal can be processed in tandem with the PVP waveform and used as an adaptive filter to remove motion components. Alternatively, a motion signal measured by one of these components can be processed and compared to a pre-existing threshold value: if the signal exceeds the pre-determined threshold value, it can indicate that the patient is moving too much to make an accurate measurement; if the signal is less than the pre-determined threshold value, it can indicate that the patient is stable and that an accurate measurement can be made.

Such circuit elements 102, 104, and 106 are typically fabricated on a small, fiberglass circuit board, such as that shown in FIG. 2E, characterized by dimensions designed to fit inside the arm-worn housing shown in FIGS. 2B and 2C.

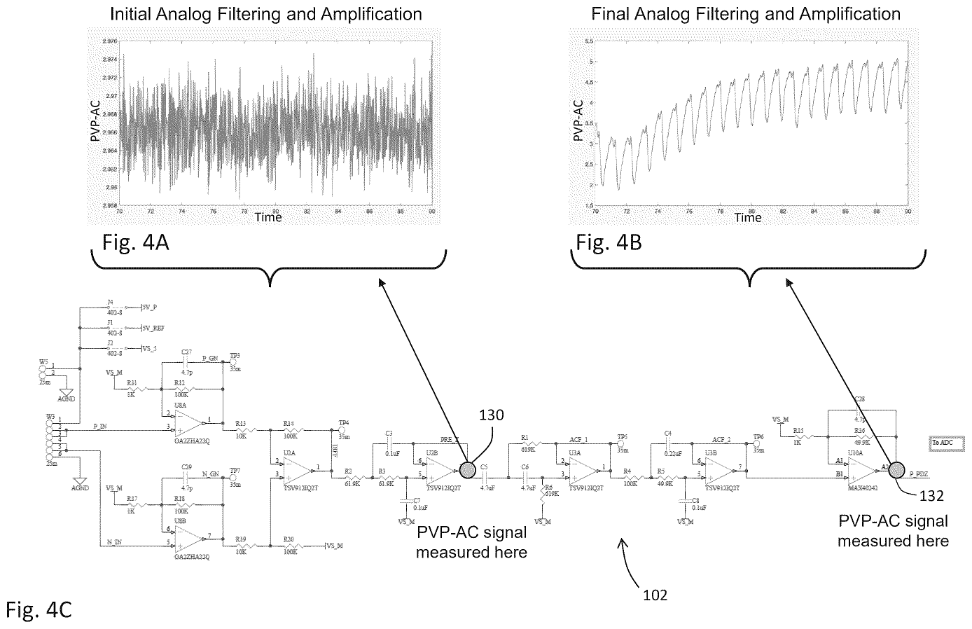

FIGS. 4A-C indicate how the circuit board 62 and associated circuit elements 102, as shown, respectively, in FIGS. 2A-C and 3, amplify and generally improve analog versions of the PVP-AC waveform. More specifically, FIG. 4A shows a time-dependent plot of the PVP-AC waveform measured at a location 130 within the circuit elements 102 corresponding to an initial analog filtering and amplification stage. As is clear from the figure, the signal-to-noise ratio of the PVP-AC waveform at this point is relatively weak, making it is difficult (if not impossible) to detect any features that correspond to actual physiological components, e.g. a heartbeat or respiration-induced pulse. In contrast, after passing through three additional amplification/filtering stages—1) differential amplifier with an additional 10× gain; 2) filter with a 25 Hz 2-pole low-pass filter and then a 0.1 Hz 2-pole high-pass filter and then a 15 Hz 2-pole low-pass filter; 3) amplifier with 50× gain—the signal is greatly improved. FIG. 4B shows the time-dependent waveform measured further down the circuit's amplifier chain at a second location 132: it features a relatively high signal-to-noise ratio and clear heartbeat-induced pulses (i.e., it shows a well-defined time-domain signal corresponding to HR). Such a waveform, when processed in the frequency domain as described above, would yield clear features corresponding to F1, thereby improving measurement of F0, F1, and their associated energies.

Importantly and as described above, the analog signal processing indicated in FIGS. 4A-C and digitization of the PVP waveform are ideally performed as close to the signal source as possible, i.e. in the arm-worn housing shown in FIGS. 2A-D. Such a configuration minimizes noise and attenuation caused by the signal propagating through a long, ‘lossy’cable (which is additionally susceptible to motion) to a remote filter/amplification circuit. Ultimately this approach yields a time-dependent waveform with the highest possible signal-to-noise ratio, thereby maximizing the accuracy to which F0, F1, and their associated energies can ultimately be determined.

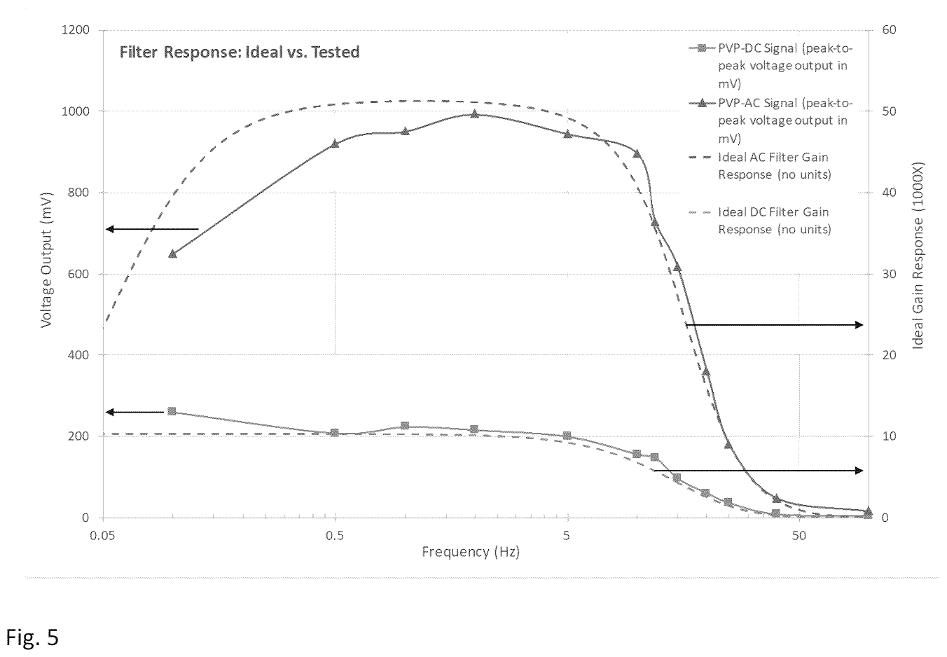

FIG. 5 shows the results of an actual experiment designed to validate the efficacy of the circuit board shown in FIG. 2E to isolate and amplify both PVP-AC and PVP-DC signals. For the experiment, a function generator and signal-reduction circuit were combined to generate input analog sinusoidal waveforms which represented PVP-AC and PVP-DC signals similar to those measured from a patient. Like actual versions of these signals, the input waveforms had frequencies ranging from 0.5-100 Hz and amplitudes in the 20 V range. In the experiment, the waveforms passed through a circuit board similar to that shown in FIG. 2E, where they were filtered and amplified according to the parameters described above (and also shown in FIG. 3), and then digitized with an analog-to-digital converter (component 106 shown in FIG. 3). The digitized waveforms were stored in memory, and peak-to-peak voltages were then calculated from the digitized signals. Finally, these values were compared to the ideal, theoretical frequency-dependent gain for the PVP-AC and PVP-DC signals, as determined with a circuit/simulator program.

As shown in FIG. 5, the measured peak-to-peak voltage outputs for the PVP-AC and PVP-DC signals are indicated by solid lines (with triangle signal markers for PVP-AC signals, and square signal markers for PVP-DC signals) and the left-hand y-axis of the graph. The ideal, theoretical gain response of the circuit board is indicated by the dashed lines and the right-hand, y-axis of the graph. The x-axis indicates logarithms of frequencies corresponding to the input sinusoidal waveforms.

FIG. 5 shows that there is strong agreement between the ideal, theoretical gain of the circuit board and the measured peak-to-peak voltages of the sinusoidal waveforms after being amplified and filtered. The agreement persists from frequencies ranging from about 0.5-50 Hz.

This indicates the circuit board shown in FIG. 2E is working as expected and effectively filtering and amplifying both PVP-AC and PVP-DC signals.

Once measured as described above, a processor analyzes PVP waveforms to determine F0, F1, and their associated energies. FIGS. 6A-G show typical time-dependent PVP-AC waveforms measured from a hospitalized patient using an IV system similar to that shown in FIG. 1. More specifically, FIG. 6A shows the waveform measured over a period of about 30 minutes. Boxes 110a, 110b, and 110c indicate 1-minute ‘waveform snippets’ that have been selected to show both the challenges of conventional PIVA sensors, and how the invention described herein is designed overcome these challenges.

FIG. 6B shows a 1-minute, time-dependent waveform snippet (i.e. w(t)) and its first time-dependent derivative (i.e. dw(t)/dt) selected over 420-480 seconds from the PVP-AC waveform in FIG. 6A, as indicated by box 110a. The waveform snippet and its derivative feature a series of heartbeat-induced pulses. Here, the derivative serves effectively as a high-pass filter that removes low-frequency components from the signal, such as those due to respiration, and amplifies high-frequency signals, such as those due to heartbeats. FIG. 6E shows the FFT of the raw, underivatized waveform snippet shown in FIG. 6B. The peaks in the figures are labeled to indicate F1 (corresponding to 70 beats/min), and the 2× and 3× harmonics of F1.

While signal components associated with F1 are readily apparent in FIGS. 6B and 6E, those associated with F0 (i.e. respiration) are absent. The patient is clearly alive and likely breathing during this 1-minute period; thus, the lack of a respiration-related signal could be due to a number of factors, such movement with the catheter, low signal associated with F0, motion-induced noise, shallow breathing, etc. In fact, a peak corresponding to F0 could be present in FIG. 6E, but simply too weak to detect without some prior knowledge of the patient's true RR. However, an independent measurement of the patient's RR, e.g. with the iPIVA physiological sensor shown in FIG. 1, would facilitate explicit and independent determination of F0. A beat-picking algorithm processing the transformed PVP waveforms could then conduct a ‘search’ in the frequency domain for F0, focusing this search around the respiratory frequency as determined by the patch sensor. This, in turn, could allow determination of both F0, F1, and their associated

energies. Alternatively, an adaptive filter could be implemented in software, wherein the filter is specifically designed to amplify signal components centered around RR, as measured with the iPIVA physiological sensor.

FIG. 6C shows a second 1-minute waveform snippet selected over 780-840 seconds from the time-dependent PVP-AC waveform in FIG. 6A, as indicated by box 110b. In this snippet, signal components due to both F0 (respiration rate) and F1 (heart rate) are more evident compared to those shown in FIGS. 6B and 6E. More specifically, heartbeat-induced pulses are clearly evident in the time domain (FIG. 6C), resulting in a well-defined F1 peak (corresponding to a heartrate of 72 beats/min) along with corresponding 2× and 3× harmonics in the frequency domain (FIG. 6F). Additionally, the respiratory component for this snippet is better defined than that shown in FIGS. 6B and 6E. Respiratory-induced undulations are clear in the time domain, resulting in a fairly well-defined F0 peak in the frequency domain, corresponding to 17 breaths/min. As with the case described above, prior knowledge of both cardiac and respiratory events as determined with the patch sensor means an algorithm informed with corresponding HR and RR values will likely have more success detecting the relevant peaks in the frequency domain. Ultimately this will improve the iPIVA sensor and any measurements made by it.

A clear example of this is shown in a third 1-minute waveform snippet selected over 1310-1370 seconds from the PVP-AC waveform shown in FIG. 6A, as indicated by box 110c. Here, signal components due to both F0 (i.e. RR) and F1 (i.e. HR) are more evident compared to those described in the previous cases. Undulations presumably corresponding to HR and RR are clear in the time domain (FIG. 6D), resulting in well-defined F0 and F1 peaks in the frequency domain (FIG. 6G). However, since the respiratory component in this snippet is so well pronounced, the F1 peak (measured at 64 beats/min) could actually correspond to a 4× harmonic of the respiratory event (4×17 breaths/min=68 breaths/min). In other words, it is not clear from simple inspection of the spectrum in FIG. 6G if the peak near 1 Hz (i.e. 60 beats/min) is due to F1 or the 4× harmonic of F0. As before, an independent measurement of HR with the patch sensor would solve this issue, as this could be used to inform determination of F1.

Features associated with F0 and F1 (e.g. their amplitude or energy) may be processed in different ways to estimate fluid-related parameters, e.g. wedge pressure and/or pulmonary arterial pressure. Further processing of the energy then yields the appropriate fluid-related parameters. Examples of such processing are described in the following references, the contents of which have been already incorporated herein by reference:
1) Hocking et al., “Peripheral venous waveform analysis for detecting hemorrhage and iatrogenic volume overload in a porcine model.”, Shock. 2016 October; 46(4): 447-52;
2) Sileshi et al., “Peripheral venous waveform analysis for detecting early hemorrhage: a pilot study.”, Intensive Care Med. 2015 June; 41(6): 1147-8;
3) Miles et al., “Peripheral intravenous volume analysis (PIVA) for quantitating volume overload in patients hospitalized with acute decompensated heart failure-a pilot study.”, J Card Fail. 2018 August; 24(8): 525-532; and
4) Hocking et al., “Peripheral i.v. analysis (PIVA) of venous waveforms for volume assessment in patients undergoing haemodialysis.”, Br J Anaesth. 2017 Dec. 1; 119(6): 1135-1140.

Parameters such as wedge pressure—as determined with both an iPIVA sensor and iPIVA physiological sensor working in concert as described herein—typically indicate the patient's fluid status, and are thus useful in managing the patient's care and resuscitating them. These parameters can be useful in the case of certain afflictions that may be treated with fluid delivery (e.g. sepsis), or those that are treated with fluid removal (e.g. heart failure). In particular, sepsis is usually treated in an intensive care unit with IV fluids and antibiotics, both of which are typically administered as soon as the condition is detected. Fluids are typically replaced so that blood pressure is maintained. Indeed, properly treating patients with fluid-related illnesses like sepsis can mean the difference between life and death. The risk of death from sepsis is as high as 30%, from severe sepsis as high as 50%, and from septic shock as high as 80%. Estimates suggest sepsis affects millions of people a year; in the developed world, approximately 0.2 to 3 people per 1000 are affected by sepsis yearly, resulting in about a million cases per year in the United States.

### iPIVA Physiological Sensor

Measurements from the iPIVA physiological sensor that directly relate to a patient's fluid status—e.g. BP, FLUIDS, SV, and CO—may complement a parameter like wedge pressure and assist in managing a patient suffering from a condition like sepsis. Sensors that measure such parameters typically deploy bio-impedance and bio-reactance measurements, operate hardware systems and algorithms similar to those described in the following pending patent applications, the contents of which are incorporated herein by reference: U.S. patent application Ser. No. 62/845,097 (filed May 8, 2019) and U.S. patent application Ser. No. 16/044,386 (filed July 24, 2018).

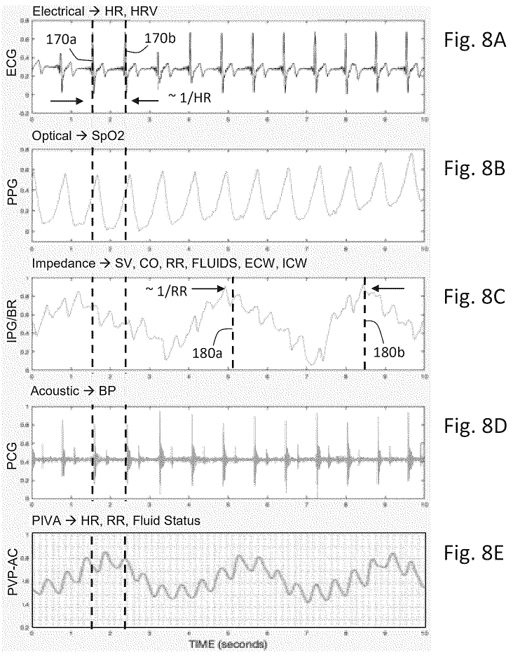

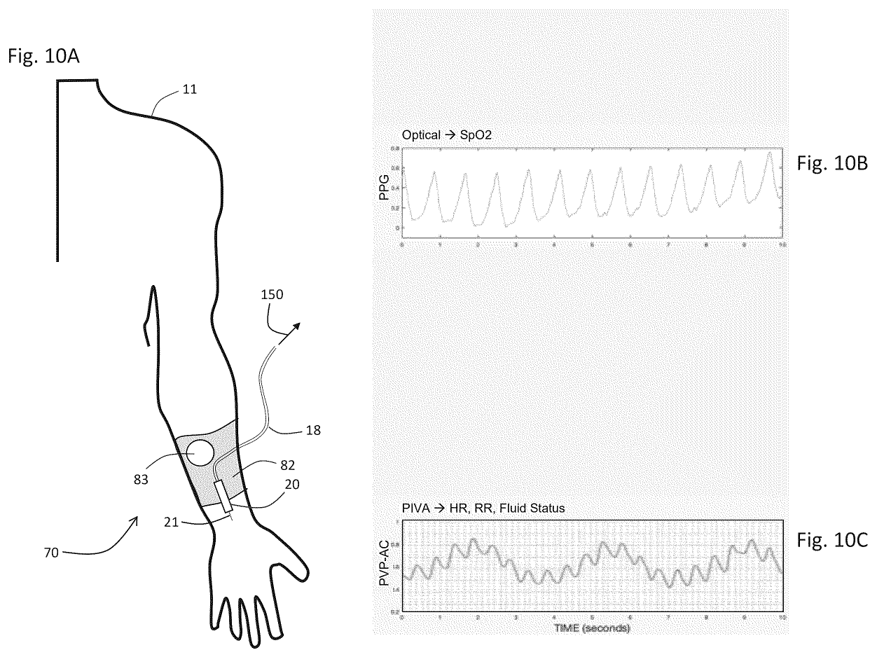

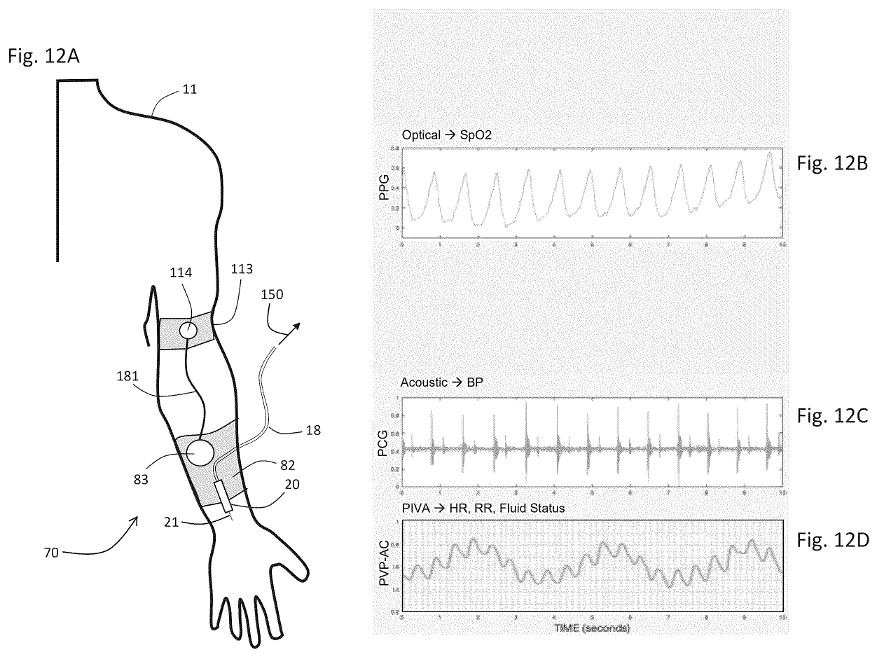

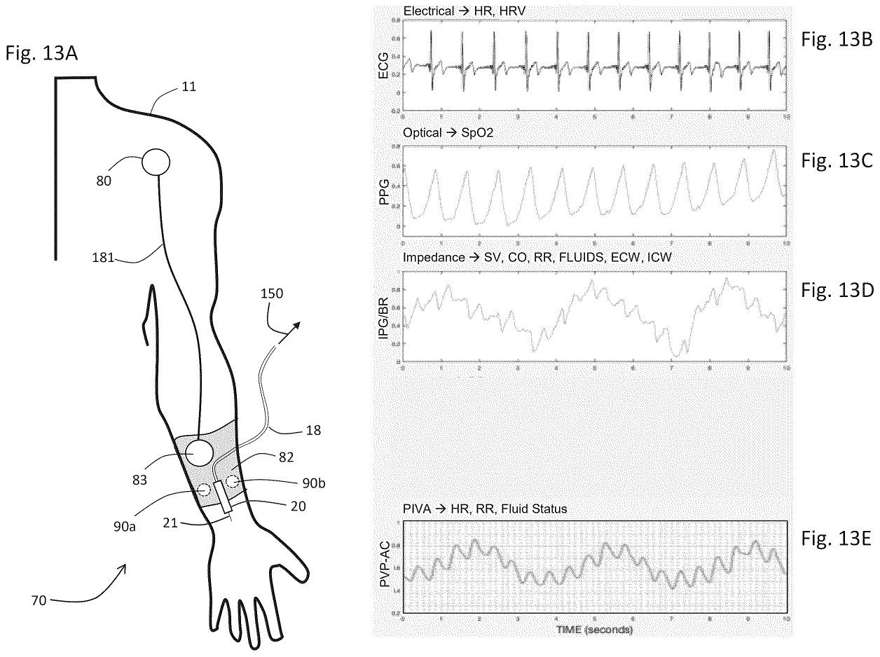

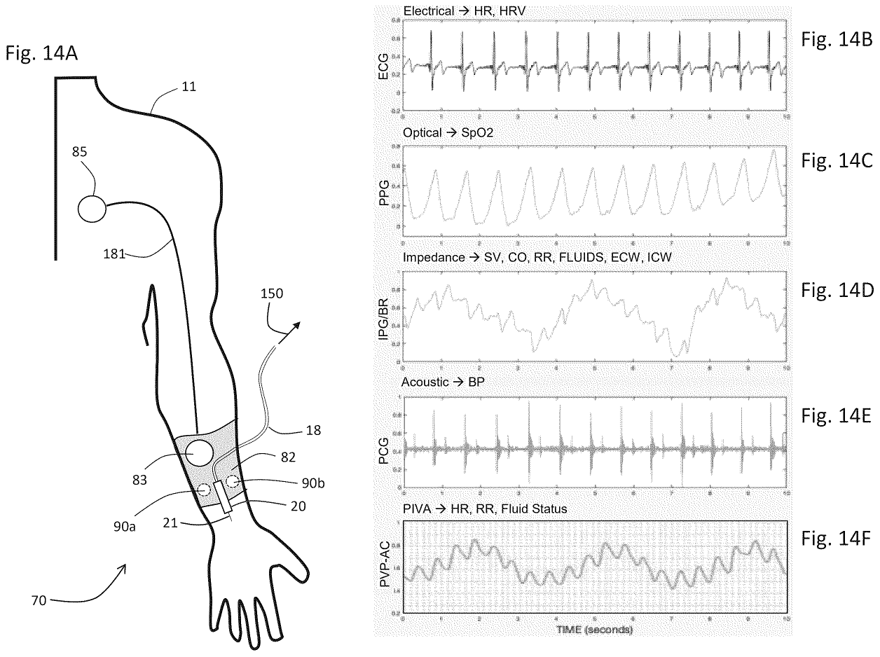

In general, and referring again to FIG. 1, a iPIVA physiological sensor 70 according to the invention typically features a central processing unit 83 that is integrated into a flexible, arm-worn wrap 82 that attaches to the patient's arm. In embodiments, such as those described in FIGS. 10-14, the arm-worn wrap 82 can include reflective or transmissive optical sensors, and one or more disposable electrodes (not shown in FIG. 1) to measure time-dependent physiological waveforms, such as those shown in FIG. 8 and 10-14, and described in more detail below. In embodiments, such as those shown in FIGS. 1 and 12-14, the arm-worn wrap 82 and central processing unit contained therein connects through a cable 81 to a secondary sensor 80, which can be worn on the patient's shoulder (as shown in FIGS. 1 and 13A), chest (as shown in FIG. 14A), or brachium (as shown in FIG. 12A). In the shoulder-worn embodiment, the secondary sensor 80 includes a pair of electrodes; these are typically adhesive, hydrogel-containing electrodes that adhere the secondary sensor 80 to the patient's skin while simultaneously measuring bio-electric signals that, with processing and when combined with a similar pair of electrodes (e.g. those in the arm-worn wrap 82), yield ECG, IPG, and BR waveforms. In the chest-worn embodiment, the secondary sensor may also include a digital microphone that measures PCG waveforms from underlying heart valves in the patient's chest, along with the pair of electrodes that function as described above. Finally, in the brachium-worn embodiment, the arm-worn wrap 82 also includes the digital microphone that measures PCG waveforms from the patient's underlying brachial artery, and pair of electrodes that function as described above.

The central processing unit 83 features a microprocessor that operates algorithms to process the waveforms, ultimately yielding parameters such as HR, HRV, RR, BP, SpO2, TEMP, SV, CO, FLUIDS. Once a measurement is complete, both the iPIVA sensor 15 and iPIVA physiological sensor transmit information (through wired and/or wireless means) to the remote processor 36, which includes a microprocessor and a display component 38. Algorithms operating through computer code running on the microprocessor in the remote processor 36 process signals from both the patch sensor 30 and iPIVA sensor 15 to determine the patient's vital signs and fluid status. For example, and as described above, an embodiment of the algorithm may use values of HR and RR determined independently by the iPIVA physiological sensor (e.g. from impedance and ECG waveforms) to inform a ‘search’ of F0 and F1 values (corresponding, respectively, to RR and HR) measured by the iPIVA sensor 15. The algorithm then determines corresponding energies of F0 and F1, and finally processes these energies to determine the patient's fluid status. Such an algorithm is indicated by the flow chart shown in FIG. 15A. Here, the search may involve using a beat-picking algorithm to process the frequency-domain spectrum (generated using one of the above-described methodologies) of a PVP waveform.

Another embodiment of the algorithm may collectively process parameters measured by the iPIVA sensor 15 (e.g. wedge pressure and blood volume, which may be correlates with energies associated with F0, F1, or some combination thereof) with those measured by the iPIVA physiological sensor 70 (e.g. BP, SpO2, FLUIDS, SV, and CO) to determine the patient's fluid status and effectively inform delivery of fluids while resuscitating the patient (e.g. during periods of sepsis and/or fluid overload). In general, by using information from both the iPIVA sensor 15 and iPIVA physiological sensor 70, a clinician can better manage the patient 11 by characterizing life-threatening conditions and help guide their resuscitation.

As a more specific example, in embodiments values of BP and SpO2 measured by the iPIVA physiological sensor can be combined with volume status determined from the iPIVA sensor to estimate a patient's blood flow and perfusion. Knowledge of these parameters, in turn, can inform estimation of how much fluid a clinician needs to deliver upon resuscitation. Similarly, SV, CO, BP, and SpO2 measured by the iPIVA physiological sensor, along with the ratio of F0 and F1 energies measured by the iPIVA sensor, each indicate a patient's level of perfusion. They can also be combined in a mathematical ‘index’ to better estimate this condition. Then these parameters or the index can be measured while the patient undergoes a technique called a ‘passive leg raise’, which is a test to evaluate the need for further fluid resuscitation in a critically ill person. The passive leg raise involves raising a patient's legs (typically without their active participation), which causes gravity to pull blood from the legs into the central organs, thereby increasing circulatory volume available to the heart (typically called ‘cardiac preload’) by around 150-300 milliliters, depending on the amount of venous reservoir. If the above-mentioned parameters or index measured by the iPIVA and patch sensors increase, this can indicate that the leg raise effectively increase perfusion in the patient's central organs, thereby indicating that they will be responsive to fluids. Clinicians can perform a similar test by providing the patient a bolus of fluids through an IV system, and then monitoring the increase or decrease in the parameters or index measured by the iPIVA and patch sensors.

In embodiments, simple linear computational methods, combined with results from clinical studies, can be used to develop models that collectively process data generated by the iPIVA sensor and iPIVA physiological sensor. In other embodiments, more sophisticated computational models, such as those involving artificial intelligence and/or machine learning, can be used for the collective processing.

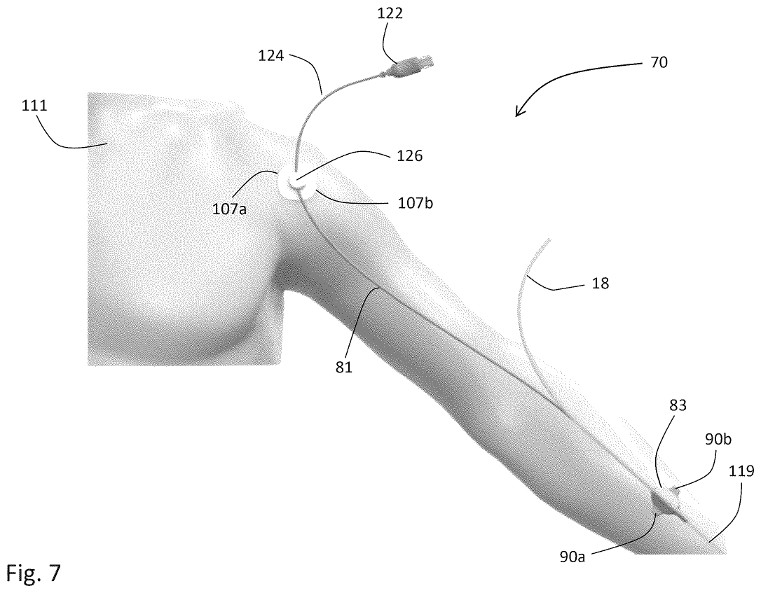

FIG. 7 shows a specific embodiment of an iPIVA physiological sensor 70 according to the invention. Such a patch 70 can integrate with a iPIVA sensor described above to serve two functions: 1) independently measure parameters such as HR and RR to better facilitate measurement of F0, F1 and their associated energies; and 2) additionally measuring parameters such as BP, FLUIDS, SV, and CO that complement parameters measured with the iPIVA sensor 15, such as wedge pressure, pulmonary arterial pressure, blood volume, and fluid status to assist in managing the patient.

The iPIVA physiological sensor 70 measures ECG, PPG, PCG, IPG, and BR waveforms from a patient, and from these calculates vital signs (HR, HRV, SpO2, RR, BP, TEMP) and hemodynamic parameters (FLUIDS, SV, and CO) as described in detail below. Once this information is determined, the patch sensor 30 wirelessly transmits it to a remote monitor so that it can be analyzed with information from the iPIVA sensor to characterize the patient.

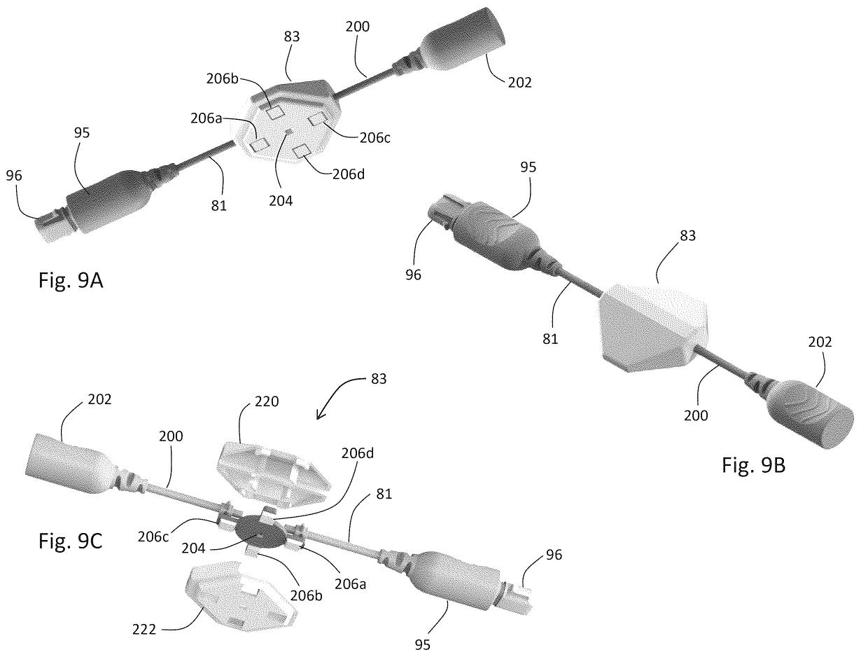

The iPIVA physiological sensor 70 shown in FIG. 7 features two primary components: 1) a central processing unit 83 worn near the patient's wrist; and 2) a secondary sensor worn 80 near the patient's left shoulder. A flexible, wire-containing cable 81 connects the central processing unit 83 and the secondary sensor 80. The central processing unit includes an optical sensor on its bottom surface (shown in more detail in FIG. 9) that measures PPG waveform from the patient's arm using a reflective-mode geometry. Electrode leads (two 90a, 90b in the central processing unit, two 107a, 107b in the secondary sensor) each connect to single-use adhesive electrodes (not shown in the figure) and help secure the iPIVA physiological sensor 70 (and particularly the optical sensor) to the patient. The central sensing/electronics module 130 features two ‘halves’139A, 139B, each housing sensing and electronic components described in more detail below, that are separated by a first flexible rubber gasket 138. Flexible circuits within the sensor 30 are typically made of a Kapton® with embedded electrical traces that connect fiberglass circuit boards (also within the sensor) within the two halves 139A, 139B of the central sensing/electronics module 130, thereby allowing the sensor to flex and conform to the patient's chest.

The electrode leads 141, 142, 147, 148 connect to a single-use electrode (not shown in the figure) and form two ‘pairs’of leads, wherein one of the leads 141, 147 in each pair injects electrical current to measure IPG and BR waveforms, and the other leads 142, 148 in each pair sense bio-electrical signals that are then processed by electronics in the central sensing/electronics module 130 to determine the ECG, IPG, and BR waveforms. Electrode leads 143, 145 also connect to a single-use electrode (also not shown in the figure), but serve no electrical function (i.e. they do not measure bio-electrical signals) and only help secure the patch sensor 30 to the patient.

IPG and BR measurements are made when the current-injecting electrodes 141, 147 inject high-frequency (e.g. 100 kHz), low-amperage (e.g. 4 mA) current into the patient's chest. In embodiments, the injected current can be sequentially adjusted to have a range of frequencies (e.g. 5-1000 kHz). In particular, low-frequency measurements (e.g. 5 kHz) typically do not penetrate cellular walls within the patient's body, and are therefore particularly sensitive to fluids disposed outside these walls, i.e. extra-cellular fluids.

The electrodes 142, 148 sense a voltage that indicates the impedance encountered by the injected current. The voltage passes through a series of electrical circuits featuring analog filters and differential amplifiers. These, respectively, filter and amplify select components of the ECG, IPG, and BR waveforms. Both the IPG and BR waveforms have low-frequency (DC) and high-frequency (AC) components that are further filtered and processed, as described in more detail below and in the references cited herein, to measure different impedance waveforms. The IPG waveform is sensitive to both phase and amplitude changes imparted on the injected current by capacitive changes (e.g. those induced by respiratory events), and conductive changes (e.g. those induced by changes in, e.g. fluids and blood flow). The BR waveform is primary sensitive to phase changes imparted on the injected current induced by these same components.

Use of a cable 134 to connect the central sensing/electronics module 130 and the optical sensor 136 allows the electrode leads (141, 142 in the central sensing/electronics module 130; 147, 148 in the secondary battery 157) can be separated by a relatively large distance when the patch sensor 30 is attached to a patient's chest. For example, the secondary battery 157 can be attached near the patient's left shoulder. Such separation between the electrode leads 141, 142, 147, 148 typically improves the signal-to-noise ratios of the ECG, IPG, and BR waveforms measured by the patch sensor 30, as these waveforms are determined from difference of bio-electrical signals collected by the single-use electrodes, which typically increases with electrode separation. Ultimately, the separation of the electrode leads improves the accuracy of any physiological parameter detected from these waveforms, such as HR, HRV, RR, BP, SV, CO, and FLUIDS.

The acoustic module 146 features a solid-state acoustic microphone that typically is a thin, piezoelectric disk surrounded by foam substrates. The foam substrates contact the patient's chest during the measurement, and couple sounds from the patient's heart into the piezoelectric disk, which then measures heart sounds from the patient. A plastic enclosure encloses the entire acoustic module 146.

The heart sounds are the ‘lub/dub’ sounds typically heard from the heart with a stethoscope: they indicate when the underlying mitral and tricuspid valves (herein “S1”, or ‘lub’ sound) and aortic and pulmonary valves (herein “S2”, or ‘dub’ sound) close (note: no detectable sounds are generated when the valves open). With signal processing, the heart sounds yield a PCG waveform that is used along with other signals to determine BP, as is described in more detail below. In other embodiments, multiple solid-state acoustic microphones are used to provide redundancy, and better detect S1, S2, heart murmurs, and other sounds from the patient's heart.

The optical sensor 136 features an optical system 160 that includes an array of photodetectors 162, arranged in a circular pattern, that surround a LED 161 that emits radiation in the red and infrared spectral regions. During a measurement, sequentially emitted red and infrared radiation from the LED 161 irradiates and reflects off underlying tissue in the patient's chest, and is detected by the array of photodetectors 162. The detected radiation is modulated by blood flowing through capillary beds in the underlying tissue. Processing the reflected radiation with electronics in the central sensing/electronics module 130 results in PPG waveforms corresponding to the red and infrared radiation, which are used to determine BP and SpO2, as described below.

The outer surface of the optical sensor 136 is covered by a heating element featuring a thin Kapton® film 165 with embedded electrical conductors arranged, e.g., in a serpentine pattern. Other patterns of electrical conductors can also be used. The Kapton® film 165 features cut-out portions that pass radiation emitted by the LED 161 and detected by the photodetectors 162 after it reflects off the patient's skin. A tab portion 167 on the thin Kapton® film 165 folds over so it can plug into the circuit board within the patch sensor 30. During use, software operating on the patch sensor 30 controls power-management circuitry on the circuit board to apply a voltage to the embedded conductors within the thin Kapton® film 165, thereby passing electrical current through them. Resistance of the embedded conductors causes the film 165 to gradually heat up and warm the underlying tissue. The applied heat increases perfusion (i.e. blood flow) to the tissue, which in turn improves the signal-to-noise ratio of the PPG waveform. A temperature sensor located on or near the Kapton® film integrates with the power-management circuitry, allowing the software to operate in a closed-loop manner to carefully control and adjust the applied temperature. Here, ‘closed-loop manner’ means that the software analyzes amplitudes of heartbeat-induced pulses the PPG waveforms, and, if necessary, increases the voltage applied to the Kapton® film 165 to increase its temperature and maximize the heartbeat-induced pulses in the PPG waveforms. Typically, the temperature is regulated at a level of between 41-42° C., which has minimal affect on the underlying tissue and is considered safe by the U.S. Food and Drug Administration (FDA).

The patch sensor 30 also typically includes a three-axis digital accelerometer and a temperature/humidity sensor (not specifically identified in the figure) to measure, respectively, three time-dependent motion waveforms (along x, y, and z-axes), humidity and TEMP values.

The patch sensor 30 typically samples time-dependent waveforms at relatively high frequencies (e.g. 250 Hz). An internal microprocessor running firmware processes the waveforms with computational algorithms to generate vital signs and hemodynamic parameters with a frequency of about once every minute. Examples of algorithms are described in the following co-pending and issued patents, the contents of which have already been incorporated herein by reference: “NECK-WORN PHYSIOLOGICAL MONITOR,” U.S. Ser. No. 14/975,646, filed Dec. 18, 2015; “NECKLACE-SHAPED PHYSIOLOGICAL MONITOR,” U.S. Ser. No. 14/184,616, filed Aug. 21, 2014; and “BODY-WORN SENSOR FOR CHARACTERIZING PATIENTS WITH HEART FAILURE,” U.S. Ser. No. 14/145,253, filed Jul. 3, 2014.

The patch sensor 30 shown in FIG. 7 is designed to maximize comfort and reduce ‘cable clutter’ when deployed on a patient, while at the same time optimizing the ECG, IPG, BR, PPG, and PCG waveforms it measures to determine physiological parameters such as HR, HRV, BP, SpO2, RR, TEMP, FLUIDS, SV, and CO. The flexible rubber gasket 138 allows the sensor 30 to flex on a patient's chest, thereby improving comfort for both male and female patients. An additional benefit of its chest-worn configuration is reduction of motion artifacts, which can distort waveforms and cause erroneous values of vital signs and hemodynamic parameters to be reported. This is due, in part, to the fact that during everyday activities, the chest typically moves less than the hands and fingers, and subsequent artifact reduction ultimately improves the accuracy of parameters measured from the patient.

### Measuring Time-Dependent Physiological Waveforms and Calculating Vital Signs and Hemodynamic Parameters

The patch sensor described above determines vital signs (HR, RR, SpO2, TEMP) and hemodynamic parameters (FLUIDS, SV, CO) by collectively processing time-dependent ECG, IPG, BR, PPG, PCG, and ACC waveforms, as shown in FIGS. 8A-E (note: BR and IPG waveforms have a similar morphology, and thus for simplicity only IPG waveforms are shown in FIG. 8D). ECG, IPG, BR, PPG, and PPG waveforms are typically characterized by a heartbeat-induced ‘pulse’; these are indicated in the figure by dashed lines 170a, 170b. The temporal separation of the pulses is inversely related to HR, as indicated in FIG. 8A. Some of the waveforms, and most notably IPG and BR waveforms, are strongly impacted by respiratory events. This is because such an event changes the capacitance—and hence impedance—in the patient's chest. Notably, FIG. 8C features undulations indicated by dashed lines 180a, 180b with a separation inversely related to RR. Values corresponding to these vital signs—HR and RR—can be used to inform a beat-picker algorithm used to locate F0 and F1 in the frequency-domain spectrum, as described in detail above.

During a measurement, embedded firmware operating on the patch sensor processes pulses in these waveforms, like those described above, with ‘beatpicking’ algorithms to determine fiducial makers corresponding to features of each pulse; these markers are then processed with additional algorithms, described herein, to determine vital signs and hemodynamic parameters.

For example, FIG. 8A shows an ECG waveform measured by the patch sensor described herein. It includes a heartbeat-induced QRS complex that informally marks the beginning of each cardiac cycle. Compared to other physiological waveforms, ECG waveforms typically have relatively good signal-to-noise ratios and are easy to analyze with beat-picking algorithms; thus, they are often used to measure HR, and QRS complexes function as ‘fiducial’ makers for analyzing some of the more complex waveforms described below. FIG. 8B shows a PPG waveform, which is measured by the optical sensor, and indicates volumetric changes in underlying capillaries caused by heartbeat-induced blood flow. As is well known in the art, the AC and DC components of PPG waveforms measured with optical radiation in the red (˜660 nm) and infrared (˜940 nm) can be collectively processed to determine values of SpO2.

The IPG waveform includes both AC and DC components: the DC component indicates the amount of fluid in the chest by measuring baseline electrical impedance; the average value of Z₀ is used to determine FLUIDS, as referenced above. The AC component which is shown in FIG. 8C, tracks blood flow in the thoracic vasculature and represents the pulsatile components of the IPG waveform. The time-dependent derivative of the AC component includes a well-defined peak that indicates the maximum acceleration of blood flow in the thoracic vasculature. Both the AC and DC components can be processed along with a parameter called left ventricular ejection time (herein “LVET”) and an equation called the Sramek-Bernstein equation (or an equivalent equation thereto) to determine SV. LVET indicates the temporal separation between the opening and closing of the aortic valves; as is known in the art, it can be determined directly from the time-dependent derivative of the AC component, or alternatively can be estimated from the HR value using a standard regression equation called Weissler's regression, or from the temporal separation of S1 and S1 peaks in the PCG waveform. CO is the mathematical product of SV and HR.

The PCG waveform shown in FIG. 8D includes two features corresponding to each heartbeat: S1 (indicating the underlying mitral and tricuspid valves closing) and S2 (indicating the aortic and pulmonary valves closing). The amplitude, timing, and frequency-domain spectra of S1 and S2 is known to be sensitive to BP. A motion waveform measured along a single axis by the accelerometer is shown in FIG. 8E. Motion waveforms are typically measured along the x, y, and z-axes, and can be used to characterize the patient's degree and type of motion, and their posture.

Parameters related to BP can be determined by analyzing the time difference between features in different waveforms. For example, algorithms operating in firmware on the patch sensor can calculate time intervals between the QRS complex and fiducial markers on each of the other waveforms. One such interval is the time separating a ‘foot’ of a pulse in the PPG waveform (FIG. 8B) and the QRS complex (FIG. 8A), referred to as pulse arrival time (herein “PAT”). PAT relates inversely to BP and systemic vascular resistance. Similarly, vascular transit time (herein “VTT”) is a time difference between fiducial markers in waveforms other than ECG, e.g. the S1 or S2 points in a pulse in the PCG waveform (FIG. 8D) and the foot of the PPG waveform (FIG. 8B). Or the peak of a pulse in the waveform (FIG. 8C) and the foot of the PPG waveform (FIG. 8B). In general, any set of time-dependent fiducials determined from waveforms other than ECG can be used to determine VTT. Collectively, PAT, VTT, and other time-dependent parameters extracted from pulses in the four physiologic waveforms are referred to as ‘systolic time intervals’, and are typically inversely related to BP.

Typically, BP-measurement methods based on systolic time intervals indicate changes in BP; they require calibration from a cuff-based system (e.g. manual auscultation or automated oscillometry) to determine absolute values of BP. Typically, such calibration methods provide initial BP values and patient-specific relationships between BP and PAT/VTT. During a cuffless measurement, the PAT/VTT values are measured in a quasi-continuous manner, and then combined with the values of BP and PAT/VTT determined during calibration to yield quasi-continuous values of BP. Such calibrations typically involve measuring the patient multiple (e.g. 2-4) times with a cuff-based BP monitor employing oscillometry, while simultaneously collecting PAT and VTT values like those described above. Each cuff-based measurement results in separate BP values. Calibrations typically last about 1 day before they need to be repeated.

In embodiments, one of the cuff-based BP measurements is coincident with a ‘challenge event’ that alters the patient's BP, e.g. squeezing a handgrip, changing posture, or raising their legs. This imparts variation in the calibration measurements, thereby improving sensitivity of the post-calibration measurements to BP swings. In other embodiments, a ‘universal calibration’ (e.g. a single calibration for all patients) can be used for the BP measurement. In other embodiments, the BP measurement is left uncalibrated, and only relative measurements of BP are calculated.

### Alternate Patch Sensors

The patch sensor described herein can have a form factor that differs from that shown in FIG. 7. For example, FIGS. 9A-B show, respectively, top and bottom images of such an alternate embodiment. Like the patch sensor described in FIG. 7, the patch sensor 230 shown in FIGS. 9A-B features two primary components: a central sensing/electronics module 252 worn near the center of the patient's chest and featuring a reflective optical sensor 274, and a secondary module 254 that connects to the central sensing/electronics module 252 with a thin cable 258. The central sensing/electronics module 252 features electrode leads 250a-d that incorporate circular magnets 251a-d that, during a measurement, connect to mated, magnetically active posts in single-use electrodes (not shown in the figure). The single-use electrodes secure the central sensing/electronics module 252 to the patient's chest. Additionally, electrode lead 250a serves as a ‘sense’ electrode to detect bioelectric signals that, after processing, yield the ECG, IPG, and BR waveforms as described above. Similarly, electrode lead 250b serves as a ‘drive’ electrode to inject high-frequency, low-amperage current into the patient's chest for the IPG and BR measurements. Electrode leads 250c-d, along with magnets 251c-d, serve no electrical function, and are simply used to better secure the sensing/electronics module 252 to the patient's chest. To complete the ECG, IPG, and BR measurements, the secondary module 254 includes a single sense electrode 256a and corresponding magnet 257a, as well as a single drive electrode 256b and corresponding magnet 257b. They form electrode pairs with sense electrode lead 250a and drive electrode lead 250b. As before, the IPG and BR waveforms can be measured at multiple frequencies ranging from about 5-1000 KHz.

The patch sensor 230 shown in FIGS. 9A and 9B, like that shown in FIG. 7, includes a reflective optical sensor 274 that features an LED 272 emitting red and infrared wavelengths. A circular array of photodetectors 270 surround the LED 272. A thin, Kapton® film 273 with embedded electrical traces surrounds the photodetectors 270 and LED 272, and generates heat when a voltage is applied; this gently warms the skin to 41° C.-42° C. using a closed-loop system, thereby increasing perfusion and amplifying the corresponding PPG waveforms.

The patch sensor 230 includes a thermally conductive metal post 264 that connects to a temperature sensor (not shown in the figure) and the patient's skin, during a measurement. With this, the patch sensor 230 can measure skin temperature. It is powered by a rechargeable Li:ion battery that can be charged through a small-scale USB port 261, or alternatively with an embedded transformer that performs wireless charging. A simple on/off switch 260 powers on the sensor 230. The sensor 230 lacks an acoustic sensor, meaning it cannot measure S1 and S2, as described above.

In other embodiments, the patch sensor 230 can have other form factors, and may include additional sensors. For example, the secondary module 254 may include an acoustic sensor, similar to the acoustic sensor (component 146) shown in FIG. 7. The reflective optical sensor 274, like the optical sensor shown in FIG. 7 (component 136), may include other, non-circular configurations of photodetectors and LEDs. For example, in embodiments, the photodetectors may be arranged in a linear, square, or rectangular arrays.

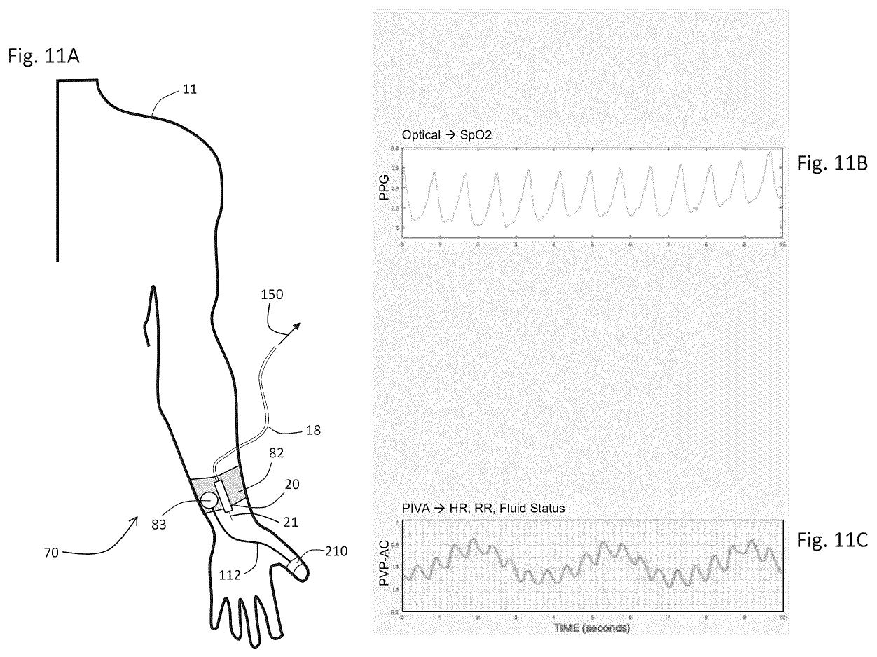

FIGS. 10-14 show alternate embodiments of the patch sensor according to the invention, along with time-dependent plots of the waveforms that they measure. In these cases, the numbered components of each patch sensor have the same function as those described in FIG. 1. For example, FIG. 10A shows an embodiment of the patch sensor 70 worn on the wrist of a patient 11. FIGS. 10B and 10C show, respectively, PPG and PVP-AC waveforms measured by the patch sensor. Here, the arm-worn wrap 82 includes a reflective optical sensor that measures the PPG waveform from the patient's wrist. FIG. 11A shows a similar embodiment of the patch sensor 70, only the optical sensor 210 is worn as a band around the thumb of the patient 11, and connects to the central processing unit 83 through a thin cable 112. For this embodiment, PPG and PVP-AC waveforms measured by the sensor are shown, respectively, in FIGS. 11B and 11C.

FIG. 12A shows a 2-part patch sensor 70 featuring an acoustic sensor 114 embedded in a band 113 wrapped around the patient's antecubital fossa. The acoustic sensor 114 connects to the central processing unit 83 through a thin cable 181, and measures PCG waveforms from acoustic sounds generated by blood pulsing through the underlying brachial artery. In this embodiment, like that shown in FIG. 10A, the optical sensor is reflective and measures PPG waveforms from the patient's wrist. Time-dependent PPG, PCG, and PVP-AC waveforms corresponding to this embodiment are shown, respectively, in FIGS. 12B-12D.

FIG. 13A shows another 2-part patch sensor 70 according to the invention. Here, an electrode-containing secondary sensor 80 is disposed near the shoulder of the patient 11, and connects to the central processing unit 83 through a cable 181. The electrode-containing secondary sensor 80 permits ECG and IPG/BR waveforms to be measured along the patient's brachial artery using a methodology similar to that described above. FIGS. 13B-13E show, respectively, the ECG, PPG, ICG/BR, and PVP-AC waveforms measured with this embodiment of the invention.

FIG. 14A shows yet another embodiment of the patch sensor 70. Like FIG. 13A, this embodiment also includes an electrode-containing secondary sensor 85. Only in this case, the secondary sensor 85 includes both electrodes and a phonocardiogram sensor that measures PPG waveforms from the underlying heart of the patient 11. Time-dependent ECG, PPG, IPG/BR, PCG, and PVP-AC waveforms measured by the patch sensor 70 are shown, respectively in FIGS. 14B-14F.

### Algorithms for Processing Signals From Both the iPIVA and Patch Sensors

FIG. 15A shows a flow chart 300 indicating the steps used by an algorithm that processes signals from both the iPIVA and patch sensors described herein to determine a parameter (e.g. wedge pressure, pulmonary arterial pressure, blood volume, fluid status) related to a patient's fluid status. FIGS. 15B-E show graphical plots corresponding to different steps listed in the flow chart 300.

The algorithm begins by explicitly determining HR/RR parameters with the patch sensor (step 320), as described above. As shown in FIG. 10B (which is taken directly from FIGS. 8A-C), for such measurements the patch sensor typically measures ECG, PPG, and/or IPG/BR waveforms, and processes them as described above to determine HR and RR. The algorithm then collects PVP waveforms in the time domain using the iPIVA sensor to generate PVP-ACₜᵢₘₑ (step 322). For this step, the algorithm may additionally include filtering algorithms (e.g. bandpass filtering) or other signal-processing techniques (e.g. an adaptive filter or averaging technique; use of an accelerometer or acoustic sensor to account for pump-induced movement and noise) to reduce or eliminate artifacts attributed to the pump. Signals are typically collected over a time period of at least several minutes. The algorithm then segments PVP-ACₜᵢₘₑ into shorter time intervals (e.g. similar to the waveform snippets shown in FIGS. 6A-D) which are classified as PVP-AC_{time, segments} (step 324). An example of PVP-ACₜᵢₘₑ is shown in FIG. 10C, with PVP-AC_{time, segments} indicated by the temporal regions of the waveforms between the dashed lines 340 in the figure. FIG. 15D shows a time-dependent plot of PVP-AC_{time, segments} corresponding to the segment indicated by the shaded circle 342; it has features indicating both heartbeat and respiratory events.

Once the algorithm generates PVP-AC_{time, segments}, each segment is transformed into the frequency domain (using, e.g., a FFT, CWT, or DWT) to generate individual frequency-domain segments classified as PVP-AC_{frequency, segments} (step 326). The algorithm then takes an ensemble average of the collection of PVP-AC_{frequency, segments} to form PVP-AC_{frequency, segments, ave} (step 328). Once PVP-AC_{frequency, segments, ave} is determined, the algorithm uses HR/RR values determined independently by the patch sensor (step 330) during step 320 to inform a peak-picking algorithm that identifies values and energies corresponding to F0 and F1 (step 332). More specifically, the algorithm uses the HR/RR values from the patch sensor as ‘truth’, and then incorporates these into a filter that prevents the algorithm for selecting erroneous peaks in the frequency-domain. Alternatively, during step 330, the HR/RR values determined from the patch sensor can be used in an adaptive filter or comparable mathematical filter to remove erroneous peaks and other features (associated, e.g., with motion or noise) from the frequency-domain spectrum, thereby making it easier to detect F0 and F1.

FIG. 15E shows plots of F0 (top plot) and F1 (bottom plot), which in this case were generated with a discrete wavelet transform. As is clear from the plots, the signal-to-noise ratio of both F0 and F1 determined using this approach is high, making it relatively easy to process parameters associated with these fiducial markers.

Once F0 and F1 are selected, their frequency is determined from the peak maximum, and their energy is determined from their peak amplitude or alternatively by integrating an area underneath the curve centered around the maximum peak amplitude (step 332). The algorithm then processes the parameters corresponding to F0 and F1, or a combination thereof, to determine a parameter related to the patient's fluid status (step 334). A clinician can then use such a parameter to treat the patient.

The algorithm indicated by step 334 in FIG. 15A can take several forms. For example, it may be a simple linear regression equation that converts parameters related to F0 and F1 measured with iPIVA (e.g. magnitude, mean, variability, phase, upslope, or downslope) to parameters related to the patient's fluid status (wedge pressure, blood volume, pulmonary arterial pressure). Here, the constants of the linear regression (slope, y-intercept) are typically determined beforehand with a clinical trial that simultaneously measures: 1) iPIVA with the system described herein; and 2) parameters related to the patient's fluid status with a reference device such as a pulmonary arterial catheter. Once these data are measured, the linear regression's slope and y-intercept can be determined by processing the information, which is then used going forward with the iPIVA measurement to determine the parameters related to the patient's fluid status. The constants of the linear regression may be grouped according to bio-metric parameters associated with the patient, such as their weight, gender, or vital signs (e.g. HR, BP). In related embodiments, the linear regression can be replaced with a more complex mathematical function, such as a polynomial, exponential, or non-linear equation, the parameters of which are determined beforehand with the above-described approach, and then used to convert iPIVA values into parameters related to the patient's fluid status.

Alternatively, a machine-learning approach can be used to develop a model that converts parameters related to F0 and F1 measured with iPIVA to those related to the patient's fluid status. One such a machine-learning approach is called a support vector machine (herein “SVM”). The approach here is similar to that used with the linear regression: data determined from a clinical trial is used to build the SVM, which is then used going forward to convert iPIVA parameters into things like cardiac wedge pressure. Other computation models that can be used in similar applications include Gaussian Kernel Functions, Boosting Ensemble, and Bagging Ensemble.

### Other Alternate Embodiments

In embodiments of the invention, algorithms operating on the iPIVA sensor can use the following steps to identify features associated with RR (i.e. F0) and HR (i.e. F1):
STEP 1) Collect a PVP waveform in the time domain, and select the desired section to process.
STEP 2) Divide the desired section of the PVP waveform in 36-second segments, and take a CWT of each segment.
STEP 3) Identify a possible value of F0 for the CWT of each segment as the median of frequencies associated with the greatest energy between 0 and 0.5 HZ. Then calculate the median F0 value for 5 consecutive segments; this becomes the working estimate of F0 for the following steps.
STEP 4) Identify the median energies at the 2nd, 3rd, and 4th harmonics of F0, as determined in STEP 3. If the energy of the 4th harmonic is the highest of the three, the frequency of the 4th harmonic becomes a candidate for F1.
STEP 5) Detect all local maxima from frequencies greater than the 4th harmonic of F0. For each maximum, count the number of other maxima with frequencies that are within 10% of a multiple of that maximum's frequency. The maximum with the highest number of multiples is the final F1 for this segment. However, if multiple peaks have the same number of multiples, or if there is only one peak, or if there are no peaks, proceed to STEP 6 below.
STEP 6) Find the frequency that is greater than the 4th harmonic of F0 and has the largest corresponding energy (i.e. the integrated area under the peak). This becomes a new candidate for F1. If there is also a candidate F1 from STEP 4, compare the energy at the two candidate F1s and choose the candidate F1 with the greater associated energy. If there is not a candidate F1 from STEP 4, the new candidate F1 is calculated as described in this STEP, and is the final F1 for this segment.

STEP 7) The median F1 from the previous 5 segments becomes the working estimate of F1.

In embodiments, variations of this approach (e.g. using an FFT or DWT in place of a CWT) can be used with the steps listed above to determine values of F0 and F1.

In other embodiments of the invention, an amplitude of either S1 or S2 (or both) heart sounds can be used to predict BP. This parameter typically increases in a linear manner with the amplitude of the heart sound. In embodiments, a universal calibration describing this linear relationship may be used to convert the heart sound amplitude into a value of BP. The algorithm for determining BP may also be based on a technique using machine learning or artificial intelligence, e.g. a technique using a SVM.

The calibration for the BP measurement, for example, may be determined from data collected in a clinical trial conducted with a large number of subjects. Here, numerical coefficients describing the relationship between BP and heart sound amplitude are determined by fitting data collected during the trial. These coefficients and a linear algorithm are coded into the sensor for use during an actual measurement. Alternatively, a patient-specific calibration can be determined by measuring reference blood pressure values and corresponding heart sound amplitudes during a calibration measurement, which proceeds an actual measurement. Data from the calibration measurement can then be fit as described above to determine the patient-specific calibration, which is then used going forward to convert heart sounds into BP values.

Time and frequency-domain analyses of IPG, BR, and PCG waveforms can be used to distinguish respiratory events such as coughing, wheezing, and to measure respiratory tidal volumes. In particular, respiratory tidal volumes are determined by integrating the area underneath a ‘respiratory pulse’ in an IPG or BR waveform (such as that indicated in FIG. 8C), and then comparing this to a pre-determined calibration. Such events may be combined with information from the iPIVA sensor to help predict patient decompensation. In other embodiments, the invention may use variations of the algorithms described above for determining vital signs and hemodynamic parameters. For example, to improve the signal-to-noise ratio of pulses within the IPG, PCG, and PPG waveforms, embedded firmware operating on the patch sensor can operate a signal-processing technique called ‘beatstacking’. With beatstacking, for example, an average pulse is calculated from multiple (e.g. seven) consecutive pulses from the IPG waveform, which are delineated by an analysis of the corresponding QRS complexes in the ECG waveform, and then averaged together. The derivative of the AC component of the IPG waveform is then calculated over a 7-sample window as an ensemble average, and then used as described above.

In other embodiments, a sensitive accelerometer can be used in place of the acoustic sensor (e.g. in the patch sensor shown in FIGS. 9A-B) to measure small-scale, seismic motions of the chest driven by the patient's underlying beating heart. Such waveforms are referred to as seismocardiogram (SCG) and can be used in place of (or in concert with) PCG waveforms to measure S1 and S2 heart sounds.

In other embodiments, signals from PIVA and iPIVA can be used to estimate conditions such as IV infiltration, extravasation, and IV occlusion. Here, changes in the time and frequency-domain PVP waveforms can indicate these conditions. For example, a gradual increase in PVP combined with a gradual reduction in F0 and F1 may indicate that an IV catheter is slipping out of the patient's vein and into surrounding tissue. Alternatively, a rapid increase in PVP coupled with a rapid elimination of F0 and F1 may indicate that the IV catheter is occluded. In other embodiments, these signals can be used to monitor IV pump performance (e.g. flow rate) or if the IV system is in a free-flow state.

These and other embodiments of the invention are deemed to be within the scope of the following claims.

What is claimed is:

1. An IV system for monitoring a patient and positioned on the patient's body, comprising:
a catheter configured to insert into the patient's venous system;
a pressure sensor connected to the catheter and configured to measure physiological signals indicating a pressure in the patient's venous system;
a motion sensor configured to measure motion signals; and,
a processing system configured to: i) receive the physiological signals from the pressure sensor; ii) receive the motion signals from the motion sensor; iii) process the motion signals by comparing them to a mathematical model to determining the patient's posture; and iv) process the physiological signals to determine a physiological parameter when the processing system determines that the patient has a pre-determined posture.

2. The system of claim 1, wherein the motion sensor is one of an accelerometer and a gyroscope.

3. The system of claim 2, wherein the motion sensor is a 3-axis accelerometer.

4. The system of claim 3, wherein the processing system is configured to calculate a motion vector by analyzing a motion signal corresponding to each axis of the 3-axis accelerometer.

5. The system of claim 1, wherein the processing system is further configured to compare the motion vector to a pre-determined look-up table to determine the patient's posture.

6. The system of claim 1, wherein the processing system is further configured to transform the physiological signals into the frequency domain to generate a frequency-domain signal.

7. The system of claim 6 wherein the processing system is configured to transform the physiological signals into the frequency domain using a FFT to generate a frequency-domain signal.

8. The system of claim 6, wherein the processing system is configured to transform the physiological signals into the frequency domain using a wavelet transform to generate a frequency-domain signal.
